SEQUENCE LISTING (15 sequences)

SEQ ID NO: 1 - protein, 23 aa, Artificial Sequence
  A synthetic sequence
SGRGKGGKGLGKGGAKRHRKVLR

SEQ ID NO: 2 - protein, 32 aa, Artificial Sequence
  A synthetic sequence
AWPFLEPVDPNDAPDYYGVIKEPMDLATMEER

SEQ ID NO: 3 - protein, 32 aa, Artificial Sequence
  A synthetic sequence
AWPFMEPVKRTEAPGYYEVIRFPMDLKTMSER

SEQ ID NO: 4 - protein, 32 aa, Artificial Sequence
  A synthetic sequence
AWPFMEPVKKSEAPDYYEVIRFPIDLKTMTER

SEQ ID NO: 5 - protein, 32 aa, Artificial Sequence
  A synthetic sequence
SWPFLEPVDESYAPNYYQIIKAPMDISSMEKK

SEQ ID NO: 6 - protein, 34 aa, Artificial Sequence
  A synthetic sequence
AWPFQQPVDAVKLNLPDYYKIIKTPMDMGTIKKR

SEQ ID NO: 7 - protein, 32 aa, Artificial Sequence
  A synthetic sequence
SAFFSFPVTDFIAPGYSMIIKHPMDFSTMKEK

SEQ ID NO: 8 - protein, 32 aa, Artificial Sequence
  A synthetic sequence
HGFFAFPVTDAIAPGYSMIIKHPMDFGTMKDK

SEQ ID NO: 9 - protein, 34 aa, Artificial Sequence
  A synthetic sequence
VQRRYYEKLTEFVADMTKIFDNCRYYNPSDSPFY

SEQ ID NO: 10 - protein, 34 aa, Artificial Sequence
  A synthetic sequence
LKNRYYVSKKLFMADLQRVFTNCKEYNPPESEYY

SEQ ID NO: 11 - protein, 34 aa, Artificial Sequence
  A synthetic sequence
LRSRYYVTRKLFVADLQRVIANCREYNPPDSEYC

SEQ ID NO: 12 - protein, 34 aa, Artificial Sequence
  A synthetic sequence
LNGGLYCTKEEFVNDMKTMFRNCRKYNGESSEYT

SEQ ID NO: 13 - protein, 34 aa, Artificial Sequence
  A synthetic sequence
LENNYYWNAQECIQDFNTMFTNCYIYNKPGDDIV

SEQ ID NO: 14 - protein, 34 aa, Artificial Sequence
  A synthetic sequence
IKNNDYQSIEELKDNFKLMCTNAMIYNKPETIYY

SEQ ID NO: 15 - protein, 34 aa, Artificial Sequence
  A synthetic sequence
IVANEYKSVTEFKADFKLMCDNAMTYNRPDTVYY

(19) United States
(12) Patent Application Publication
(10) Pub. No.: US 2023/0391730 A1
(43) Pub. Date: Dec. 7, 2023

(54) INHIBITORS OF THE BROMODOMAIN PHD FINGER TRANSCRIPTION FACTOR (BPTF) AS ANTI-CANCER AGENTS

(71) Applicant: Regents of the University of Minnesota, Minneapolis, MN (US)

(72) Inventors: William Pomerantz, Minneapolis, MN (US); Huda Zahid, Minneaplis, MN (US); Caroline Buchholz, Bloomington, MN (US)

(21) Appl. No.: 18/248,364

(22) PCT Filed: Oct. 7, 2021
(86) PCT No.: PCT/US2021/054040

§ 371 Date: Apr. 7, 2023

Related U.S. Application Data
(60) Provisional application No. 63/089,999, filed on Oct. 9, 2020.

(51) Int. Cl. C07D 237/14; C07D 401/12; C07D 403/12; A61K 31/501; A61K 31/50; A61K 45/06; A61P 35/00

(52) U.S. Cl. CPC C07D 237/14; C07D 401/12; C07D 403/12; A61K 31/501; A61K 31/50; A61K 45/06; A61P 35/00

(57) ABSTRACT

The disclosure relates to a compound of the formula (I) or a pharmaceutically acceptable salt thereof; wherein: X1 is P, NR5 or S, wherein R5 is H, alkyl, arylalkyl or OR6, wherein R6 is H, alkyl, or arylalkyl; R1 and R2 are each independently H, alkyl, alkynyl, cycloalkyl or heterocyclyl; R3 is halo (e.g., Cl and Br); and R4 is —NHR7, wherein R7 is aryl, arylalkyl, heterocyclyl or heterocyclylalkyl; or R4 is halo; and R3 is —NHR7, wherein R7 is aryl, arylalkyl, heterocyclyl or heterocyclylalkyl.

### CROSS-REFERENCE TO RELATED APPLICATIONS

This application claims the benefit of U.S. Provisional Appl. Ser. No. 63/089,999, filed Oct. 9, 2020, which is incorporated by reference as if fully set forth herein.

### STATEMENT OF U.S. GOVERNMENT SUPPORT

This invention was made with government support under grants R01GM121414-04 and R35 GM140837-01 awarded by the National Institutes of Health. The government has certain rights in the invention.

### BACKGROUND

Epigenetic processes involve heritable changes in gene expression without altering the underlylng DNA sequence. Gene accessibility leading to these changes occurs through mechanisms such as DNA methylation, co-valent modifications of histones, chromatin remodeling, and exchange of histones. In the case of chromatin remodeling, ATP-dependent processes are catalyzed by multidomain protein complexes which include SWI/SNF, ISWI, CHD and INO80. Of these, SWI/SNF has been extensively studied and is implicated in ˜20% of human cancers. The mammalian SWI/SNF complexes, BAF and PBAF, have emerged as attractive epigenetic therapeutic targets, for which chemical inhibitors and catalytically degrading molecules of complex members BRD7 and BRD9 have been developed. In contrast, the ISWI family is less well-studied for its potential role as a therapeutic target. Nucleosome Remodeling Fac-tor (NURF) is one member of the ISWI family, consisting of an ATPase domain SNF2L, a WD-repeat protein RbAP46/48, and a chromatin-binding protein, BPTF (FIG. 1A). Chemical probe development for these complex members remains at an early stage. BPTF (Bromodomain PHD Finger Transcription Factor) is the largest subunit of NURF and is considered essential for its function. BPTF contains a bromodomain, two PHD fingers, a DNA-association domain, three nuclear receptor binding motifs, and a glutamine-rich domain. Both the bromodomain and C-terminal PHD domain are structurally well-characterized and are responsible for binding to acetylated and methylated histones respectively. While BPTF is known to be essential in normal cellular processes such as embryonic development, T-cell homeostasis and differentiation of mammary epithelial cells, the oncogenic effects of BPTF have been recently well-documented. BPIF is overexpressed in melanoma, where it impacts MAPK signaling, and is regulated by the melanocyte-inducing transcription factor, MITF. High BPTF levels correlate with c-Myc expression in various cancers, regulation of Myc signaling, and Myc protein-protein intractions. Additional oncogenic roles for BPTF have been found in breast cancer, non-small-cell lung cancer, colorectal cancer, and high-grade gliomas.

BPTF also confers chemoresistance to cancer cells; overexpression of BPTF promotes resistance to BRAF inhibitors in melanoma and knockdown of BPTF sensitizes hepatocellular carcinoma cells to chemotherapeutic drugs. The implication of BPTF in cancer and its key role as a NURF subunit makes it a potential new therapeutic target for small molecule inhibitor development. One attractive targeting element is the bromodomain, which is computationally predicted to be highly druggable. However, the role of the bromodomain in many of these disease states needs to be established.

While inhibitor development for class II bromodomain and extraterminal domain (BET) family proteins (FIG. 1B) have resulted in translation of numerous inhibitors into the clinic, non-BET class 1 bromodomains such as BPTF have received less attention. AU1 has been reported as the first small-molecule inhibitor of the BPTF bromodomain (K_{d}=2.8 μM) (FIG. 1C).

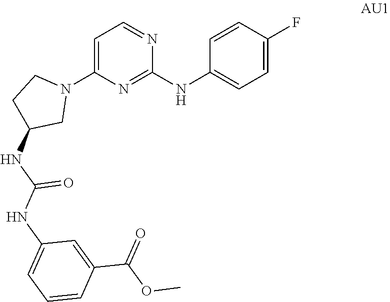 [SMILES: COC(=O)c1cccc(NC(=O)N[C@H]2CCN(c3ccnc(Nc4ccc(F)cc4)n3)C2)c1]

Importantly this molecule was selective over the BET protein BRD4, given the strong phenotype of BRD4 in regulating cell cycle, proliferation, and inflammatory pathways. AU1 has since been used in mouse mammary epithelial cells showing decreased proliferation, cell cycle arrest, and reduced c-Myc-DNA occupancy; however in other cell lines, off-target activity was identified. Most recently, AU1 showed enhancement of anti-cancer activity when used in combination with the chemotherapeutic drug doxorubicin in vitro and in vivo in 4T1 breast cancer models. Mechanistic studies showed these processes to be autophagy-dependent and AU1 effects on topo2-isomerase-DNA crosslinks and DNA damage recapitulated the effects from BPTF knockdown experiments. However, the off-target kinase activity of AU1, its poor physicochemical properties, and low ligand efficiency, posed significant challenges to inhibitor development and highlighted the need for new and more potent BPTF inhibitors.

Recently, several inhibitors were disclosed online by the structural genomics consortium; TP-238, a dual CECR2/BPTF chemical probe (12-fold higher affinity for CECR2 over BPTF) and NVS-BPTF-1, a potent BPTF inhibitor in vitro but with poor solubility and ADME properties.

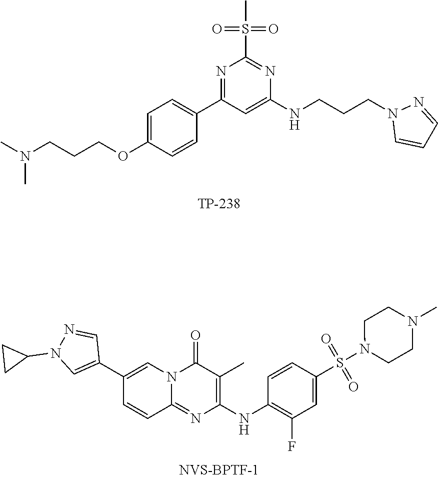 [SMILES: CN(C)CCCOc1ccc(-c2cc(NCCCn3cccn3)nc(S(C)(=O)=O)n2)cc1 ; Cc1c(Nc2ccc(S(=O)(=O)N3CCN(C)CC3)cc2F)nc2ccc(-c3cnn(C4CC4)c3)cn2c1=O]

Encouragingly, TP-238 administration to cells was shown to reduce BPTF chromatin binding, supporting the importance of bromodomain inhibition. However, detailed reports and theft characterization have yet to be described in the primary literature. Given the emerging role of BPTF in cancer, there is a significant need for improved potent and selective inhibitors.

### BRIEF DESCRIPTION OF THE FIGURES

The drawings illustrate generally, by way of example, but not by way of limitation, various embodiments discussed herein.

FIGS. 1A-1C are A) BPTF interacts with chromatin through the bromodomain (BRD) and PHD domain, directing the chromatin remodelling complex NURF to genes, leading to downstream phenotypic effects such as Myc regulation, MAPK signaling and resistance to chemo-therapeutics. B) Part of the bromodomain phylogenetic tree, showing class I and class II (BET) bromodomains. C) Reported BPTF bromodomain inhibitors with in vitro affinities.

FIG. 2 is a cocrystal structure of GSK4027 (cyan) with BPTF bromodomain (gray, PDB: 7K6R). Four conserved structured waters are shown as red spheres. Hydrogen bonds are shown as yellow dashed lines and aromatic interaction as orange dashed line. The distances (Å) between key residues are indicated. Inset: Residues in other class I bromodomains (PCAF, GCN5 and CECR2) corresponding to D2957 and D2960 in BPTF.

FIGS. 3A-3E are BPTF bromodomain (gray) cocrystal structures with A,B) 10 (magenta, PDB: 7RWP, 1.73Å resolution), C) 12 (yellow, PDB: 7RWQ, 1.90Å resolution), D) 13 (orange, PDB: 7RWO, 1.58Å resolution) and E) 19 (blue, PDB ID: 7M2E, 1.75Å resolution). Hydrogen bonds are shown as yellow dashed lines. The distances (Å) between key residues are indicated. Three of the conserved structured waters are excluded for clarity.

FIGS. 4A-4F are A) The tryptophan residues in the binding sites of BPTF (PDB ID: 7JT4), PCAF, CECR2 and BRD4(1) were fluorine-labeled to act as reporters for PrOF NMR. BZ1 was titrated with 50 μM of 5-fluorotryptophan (5FW)-labeled proteins. Slow chemical exchange regimes were observed with B) 5FW-BPTF and C) 5FW-PCAF, indicating the high affinity of BZ1 for these proteins. Intermediate exchange with D) 5FW-CECR2 and E) 5FW-BRD4(1) indicated BZ1 was a weaker binder. F) Affinity values of BZ1 for BPTF (blue) and BRD4(1) (red) were quantified using AlphaScreen competition experiments.

FIGS. 5A-5C are A) Single-point measurement of 140 nM BZ1 against a representative panel of 32 bromodomains via BROMOscan. Percent inhibition ranges are shown by: circles 95-100%, triangles 90-95% and squares 65-90%. (Adapted with permission from Pomerantz et al.) 11.B) Kd values for BZ1 with BPTF and off-target class I (PCAF, GCN5L2, CECR2,) and class IV (BRD7, BRD9) bromodomains and BRD4(1) as the highest off-target from the BET family and Kd values for compound 21, 22 and 24 with BPTF, PCAF and BRD9. Values are averages of two technical replicates, N=1, except BZ1 with BPTF and BRD9, which are averages of two experimental replicates. C) Sequence alignment (SEQ ID NOs: 2-15) of selected bromodomains highlighting WPF shelf motif (cyan), 3D equivalents of acidic triad (yellow), Kac mimetic H-bonding groups (magenta), and the gatekeeper residue (green).

FIGS. 6A-6D show how AU1, 19 and BZ1 synergize with chemotherapy drug doxorubicin in 4T1 breast cancer cells. Compound 20 was used as a negative control. 4T1 cells were tested A) without doxorubicin B) in the presence of 50 nM doxorubicin. As a control for off-target effects, shRNA-mediated BPTF knockdown (KD) cells were treated with BPTF inhibitors with and without doxorubicin in C) and D) respectively. Fraction survival values are averages of three experimental replicates, except DMSO controls which are averages of nine experimental replicates.

FIGS. 7A-7C show RT-qPCR Analysis of BPTF Regulated Genes. A) Sfn: DMSO vs AU1: p=0.5994, 19 vs 20: p=0.0263*, DMSO vs 19: p=0.0388*, DMSO vs 20: p=0.9798 B) Sprr1a: DMSO vs AU1: p=0515 NS, 19 vs 20: p=0.3264 NS, DMSO vs 19: p=0.8727 NS, DMSO vs 20: p=0.9037 NS C) Myc: DMSO vs AU1: p>0.9999 NS, 19 vs 20: p=0.0568, DMSO vs 19: p=0.3265 NS, DMSO vs 20: p=0.9557 NS.

### SUMMARY

The compounds described herein generally relate to BPTF bromodomain inhibitors, such as the compound referred to herein as BZ1, which has nanomolar affinity (K_{d}=6.3 nM) and >350-fold selectivity over BET bromodomains.

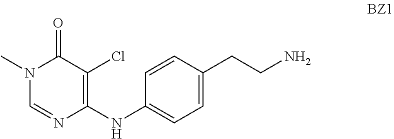 [SMILES: Cn1cnc(Nc2ccc(CCN)cc2)c(Cl)c1=O]

Inhibitors such as BZ1 are obtained via a facile synthesis routes. The high affinity, aqueous solubility, and physicochemical properties of BZ1 enabled accessing cocrystal structures with BPIF for rationalizing structure-activity-relationship data and to identify an acidic triad as a targetable feature of the binding site. Finally, 4T1 breast cancer cell chemotherapeutic synergy model previously validated for BPTF on-target engagement, to show that the compounds described herein are both generally well-tolerated by cells, and enhance doxorubicin cytotoxic effects to wild type breast cancer cells but not identical cells with BPIF knockdown, demonstrating specificity in their biological activity.

### DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

The disclosure generally relates to compounds of the formula (I):

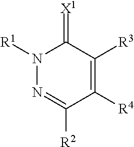

or a pharmaceutically acceptable salt thereof;
wherein:
X¹ is O, NR⁵ or S, wherein R⁵ is H, alkyl, arylalkyl or OR⁶, wherein R⁶ is H, alkyl, or arylalkyl;
R¹ and R² are each independently H, alkyl, cycloalkyl or heterocyclyl;
R³ is halo (e.g., Cl and Br); and
R⁴ is —NHR⁷, wherein R⁷ is aryl, arylalkyl, heterocyclyl or heterocyclylalkyl; or
R⁴ is halo; and
R³ is —NHR⁷, wherein R⁷ is aryl, arylalkyl, heterocyclyl or heterocyclylalkyl. In some instances, when R³ is chloro, R⁷ is not pyrrolidinyl or piperidinyl.

Compounds of the formula (I) include compounds wherein R³ or R⁴ can be —NHR⁷, wherein can be, for example, heterocyclyl, such as a four-, five- or six-membered heterocyclyl group, wherein the heterocyclyl group is selected from the group consisting of azetidinyl, tetrahydrofuranyl, furanyl, thetrahydrothiophenyl, thiophenyl, imidazolyl, diazolyl, 1,2,3-triazolyl, 1,2,4-triazolyl, thiazolyl, oxazolyl, morpholinyl, pyrrolidinyl, piperidinyl, piperazinyl, and the like, each of which can be substituted or unsubstituted. In some instances, such as when R³ is chloro, then R⁷ is azetidinyl, tetrahydrofuranyl, furanyl, thetrahydrothiophenyl, thiophenyl, imidazolyl, diazolyl, 1,2,3-triazolyl, 1,2,4-triazolyl, thiazolyl, oxazolyl, morpholinyl, and piperazinyl.

Thus, for example compounds of the formula (I) include compounds of the formula (Ia) and (Ib):

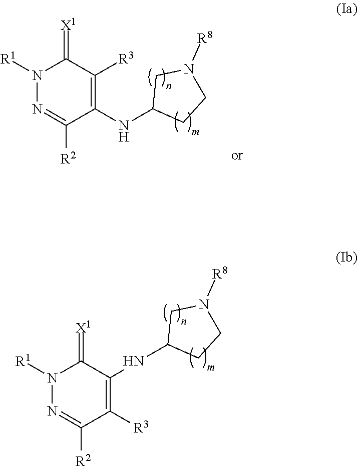

or a pharmaceutically acceptable salt thereof;
wherein R⁸ is H, alkyl or arylalkyl;
m is 0, 1, 2 or 3; and
m is 0, 1, 2 or 3, such that m+n can be 2, 3 or 4. Examples of compounds of the formulae (Ia) and (Ib) are compounds wherein n is 1 and m is 0, 1, 2 or 3, such that m+n can be 1, 2, 3 or 4. For example, compounds of the formulae (Ia) and (Ib) include compounds of the formulae:

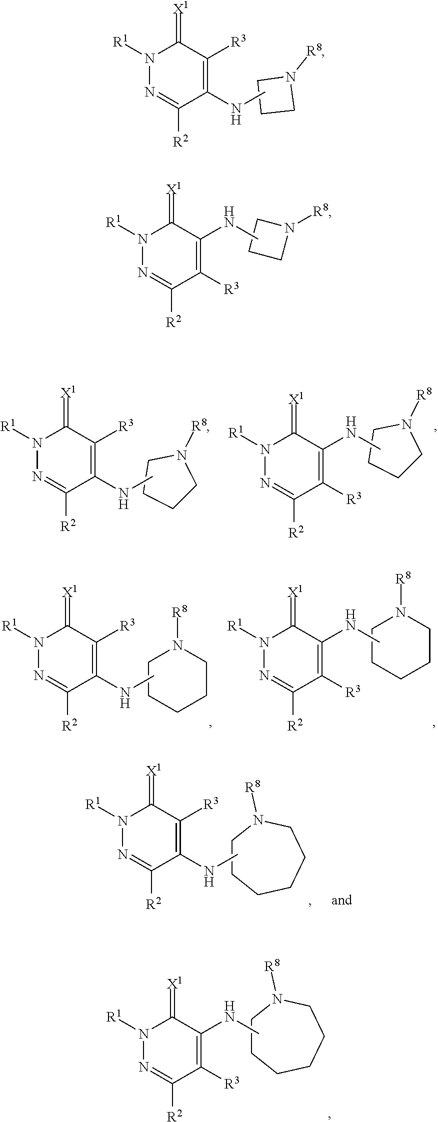

or a pharmaceutically acceptable salt thereof, such as compounds of the formulae:

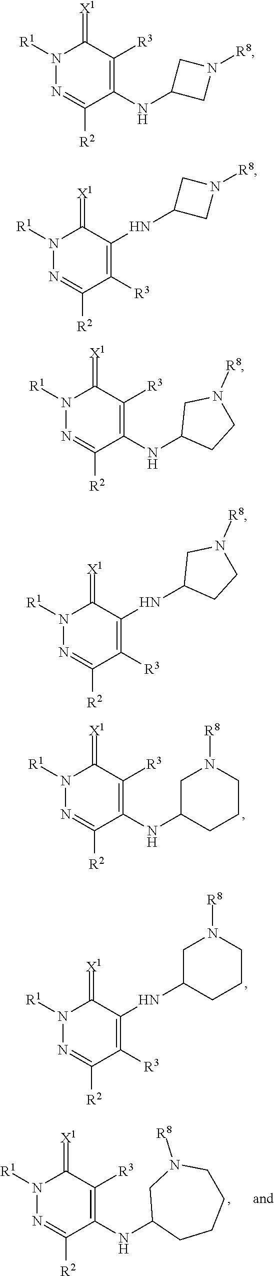

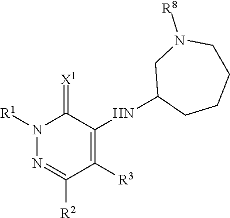

or a pharmaceutically acceptable salt thereof.

Thus, examples of compounds of the formulae (I) include compounds wherein R¹ is alkyl (e.g., C₁-C₆-alkyl, C₁-C₃-alkyl, including methyl, ethyl, propyl, butyl, and the like) or cycloalkyl (e.g., C₃-C₆ cycloalkyl, such as cyclopropyl, cyclobutyl, cyclopentyl, and cyclohexyl). Alternatively, or in addition to having such R¹ groups, compounds of the formula (I) include compounds wherein R² is H. Alternatively, or in addition to having such R¹ and/or R² groups, compounds of the formula (I) include compounds wherein R⁸ is H or alkyl (e.g., C₁-C₆-alkyl, C₁-C₃-alkyl, including methyl, ethyl, propyl, butyl, and the like).

Compounds of the formula (I) include compounds wherein R³ or R⁴ can be —NHR⁷, wherein R⁷ can be, for example, aryl or arylalkyl and the aryl group of the aryl or arylalkyl group can be substituted or unsubstituted. Thus, for example, R⁷ can be substituted or unsubstituted mono- and polycyclic (C₆-C₂₀)aryl groups, including fused and non-fused polycyclic (C₆-C₂₀)aryl groups and substituted or unsubstituted mono- and polycyclic (C₆-C₂₀)aryl alkyl groups, including fused and non-fused polycyclic (C₆-C₂₀)aryl alkyl groups. Examples of such compounds include compounds of the formulae (Ic) and (Id):

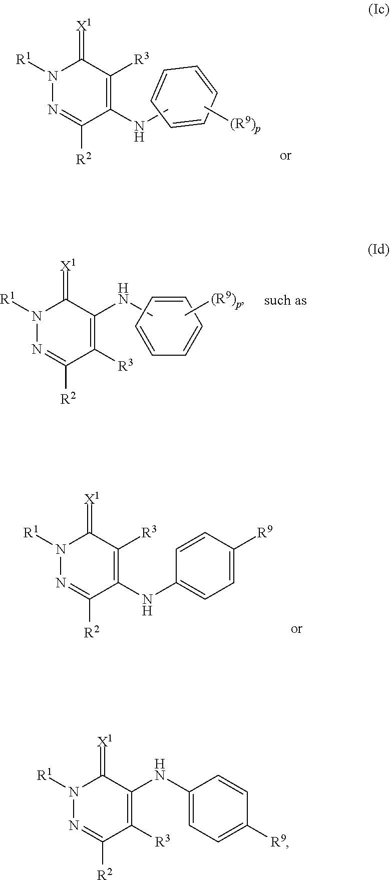

or a pharmaceutically acceptable salt thereof;
wherein:
p is 1, 2 or 3; and
each R⁹ is H or a substituent. For example, each R⁹ is independently H or a substituent, such as H, alkyl, alkoxy, amino, aminoalkyl, amido, amidoalkyl or two R⁹ groups located on adjacent carbon atoms can, together with the atoms to which they are attached, form a cyclic group, such as a heterocyclyl or a cycloalkenyl group, such that R⁷ is a group of the formula:

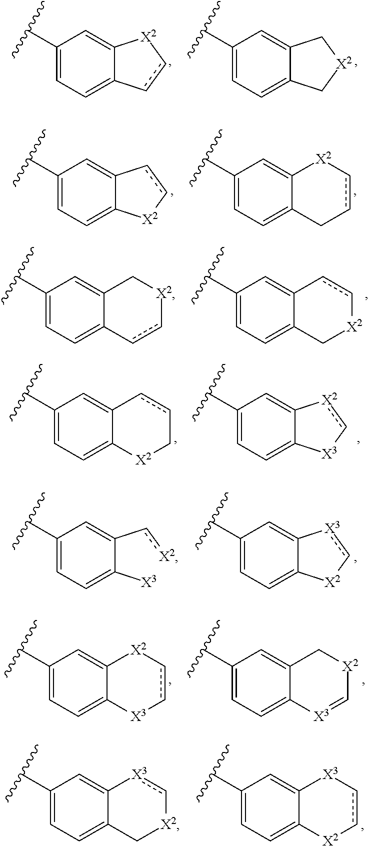

wherein the dashed line can represent a double bond; X² is CH₂, O or NR¹⁰, wherein R¹⁰ is absent when a double bond is present; and X³ is CH₂, O or NR¹⁰. Thus, for example, R⁷ can be groups of the formulae:

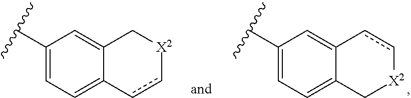

such as groups of the formulae;

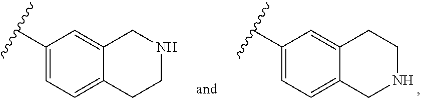 [SMILES: *c1ccc2c(c1)CNCC2 ; *c1ccc2c(c1)CCNC2]

respectively;

Compounds of the formula (I) also include compounds of the formulae (Ie) and (If):

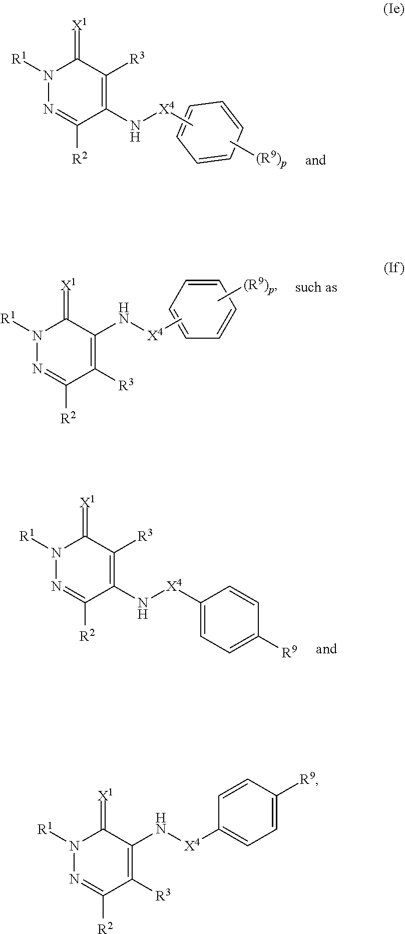

or a pharmaceutically acceptable salt thereof;
wherein:
X⁴ is alkyl (for example CH₂);
p is 1, 2 or 3; and
each R⁹ is H or a substituent. For example, each R⁹ is independently H or a substituent, such as H, alkyl, alkoxy, amino, aminoalkyl, amido, amidoalkyl or two R⁹ groups located on adjacent carbon atoms can, together with the atoms to which they are attached, form a cyclic group, such as a heterocyclyl or a cycloalkenyl group, such that R⁷ is a group of the formula:

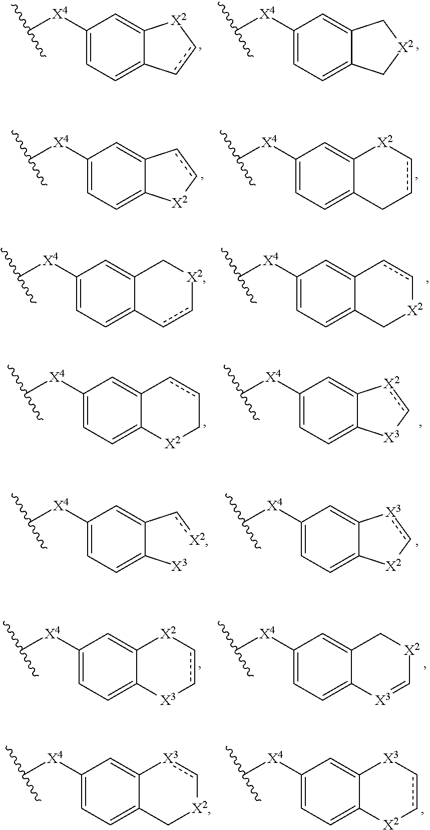

wherein the dashed line can represent a double bond; X² is CH₂, O or NR¹⁰, wherein: R¹⁰ is absent when a double bond is present; and X³ is CH₂, O or NR¹⁰. Thus, for example, R⁷ can be groups of the formulae:

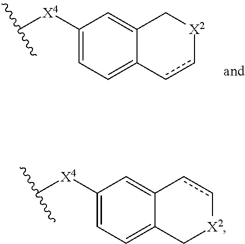

such as groups of the formulae:

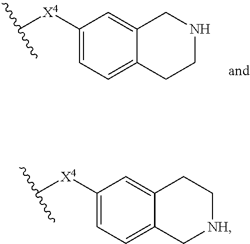

respectively.

Examples of compounds of the formula (I) include compounds of the formulae:

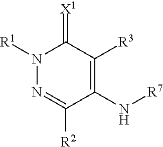

| R¹ | R² | R³ | X¹ | R⁷ |
|---|---|---|---|---|
| CH₃ | H | Cl | O | 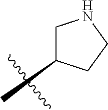 |
| CH₃ | H | Cl | O | 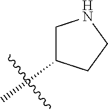 |
| CH₃ | H | Cl | O | 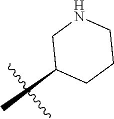 |
| CH₃ | H | Cl | O | 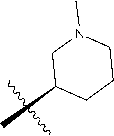 |
| CH₃ | H | Cl | O | 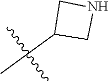 [SMILES: *C1CNC1] |
| CH₃ | H | Cl | O | 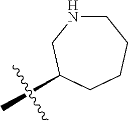 |
| CH₃ | H | Cl | O | 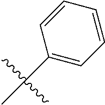 [SMILES: *c1ccccc1] |
| CH₃ | H | Cl | O | 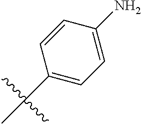 [SMILES: *c1ccc(N)cc1] |
| CH₃ | H | Cl | O | 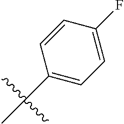 [SMILES: *c1ccc(F)cc1] |
| CH₃ | H | Cl | O | 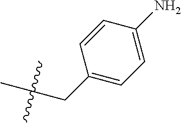 [SMILES: *Cc1ccc(N)cc1] |
| CH₃ | H | Cl | O | 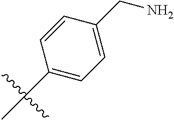 [SMILES: *c1ccc(CN)cc1] |
| CH₃ | H | Cl | O | 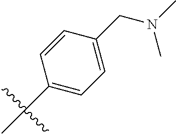 [SMILES: *c1ccc(CN(C)C)cc1] |
| CH₃ | H | Cl | C | 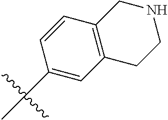 [SMILES: *c1ccc2c(c1)CCNC2] |
| CH₃ | H | Cl | O | 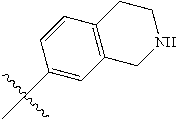 [SMILES: *c1ccc2c(c1)CNCC2] |
| CH₃ | H | Cl | O | 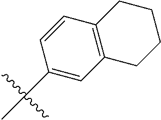 [SMILES: *c1ccc2c(c1)CCCC2] |
| CH₃ | H | Cl | O | 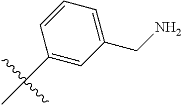 [SMILES: *c1cccc(CN)c1] |
| CH₃ | H | Cl | O | 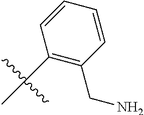 [SMILES: *c1ccccc1CN] |
| CH₃ | H | Cl | O | 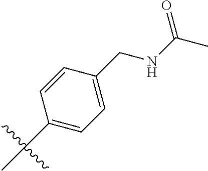 [SMILES: *c1ccc(CNC(C)=O)cc1] |
| CH₃ | H | Cl | O | 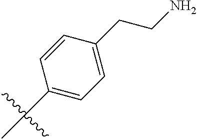 [SMILES: *c1ccc(CCN)cc1] |
| CH₃ | H | Cl | O | 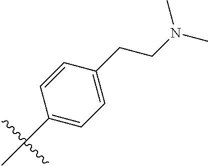 [SMILES: *c1ccc(CCN(C)C)cc1] |
| CH₃ | H | Br | O | 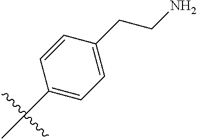 [SMILES: *c1ccc(CCN)cc1] |
| CH₃ | H | Cl | O | 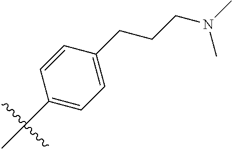 [SMILES: *c1ccc(CCCN(C)C)cc1] |
| CH₃CH₂ | H | Cl | O | 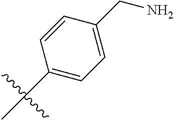 [SMILES: *c1ccc(CN)cc1] |
| 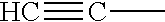 | H | Cl | O | 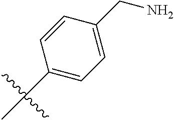 [SMILES: *c1ccc(CN)cc1] |
| (CH₃)₂CH— | H | Cl | O | 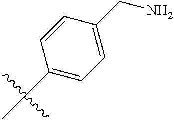 [SMILES: *c1ccc(CN)cc1] |

Examples of compounds of the formula (I) include compounds of the formulae:

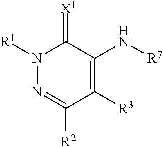

| R¹ | R² | R³ | X¹ | R⁷ |
|---|---|---|---|---|
| CH₃ | H | Cl | O | 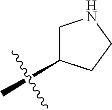 |
| CH₃ | H | Cl | O | 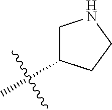 |
| CH₃ | H | Cl | O | 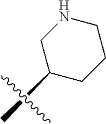 |
| CH₃ | H | Cl | O | 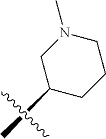 |
| CH₃ | H | Cl | O | 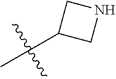 [SMILES: *C1CNC1] |
| CH₃ | H | Cl | O | 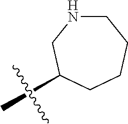 |
| CH₃ | H | Cl | O | 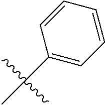 [SMILES: *c1ccccc1] |
| CH₃ | H | Cl | O | 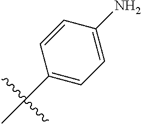 [SMILES: *c1ccc(N)cc1] |
| CH₃ | H | Cl | O | 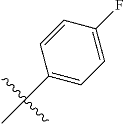 [SMILES: *c1ccc(F)cc1] |
| CH₃ | H | Cl | O | 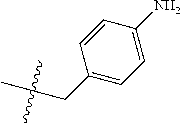 [SMILES: *Cc1ccc(N)cc1] |
| CH₃ | H | Cl | O | 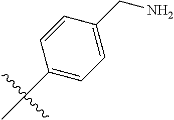 [SMILES: *c1ccc(CN)cc1] |
| CH₃ | H | Cl | O | 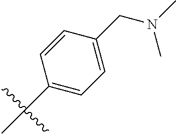 [SMILES: *c1ccc(CN(C)C)cc1] |
| CH₃ | | Cl | O | 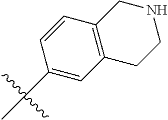 [SMILES: *c1ccc2c(c1)CCNC2] |
| CH₃ | H | Cl | O | 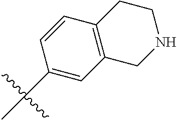 [SMILES: *c1ccc2c(c1)CNCC2] |
| CH₃ | H | Cl | O | 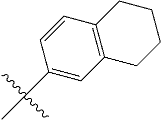 [SMILES: *c1ccc2c(c1)CCCC2] |
| CH₃ | H | Cl | O | 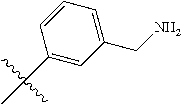 [SMILES: *c1cccc(CN)c1] |
| CH₃ | H | Cl | O | 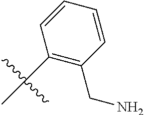 [SMILES: *c1ccccc1CN] |
| CH₃ | H | Cl | O | 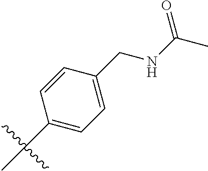 [SMILES: *c1ccc(CNC(C)=O)cc1] |
| CH₃ | H | Cl | O | 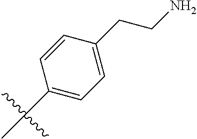 [SMILES: *c1ccc(CCN)cc1] |
| CH₃ | H | Cl | O | 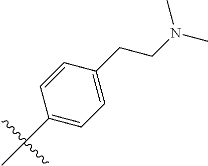 [SMILES: *c1ccc(CCN(C)C)cc1] |
| CH₃ | H | Br | O | 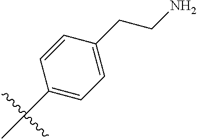 [SMILES: *c1ccc(CCN)cc1] |
| CH₃ | H | Cl | O | 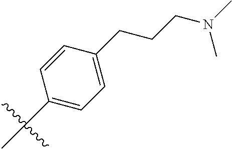 [SMILES: *c1ccc(CCCN(C)C)cc1] |
| CH₃CH₂ | H | Cl | O | 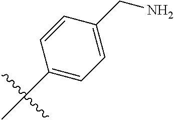 [SMILES: *c1ccc(CN)cc1] |
| 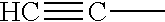 | H | Cl | O | 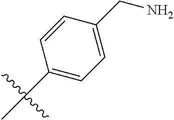 [SMILES: *c1ccc(CN)cc1] |
| (CH₃)₂CH— | H | Cl | O | 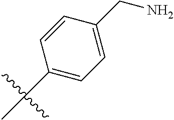 [SMILES: *c1ccc(CN)cc1] |

This disclosure also contemplates pharmaceutical compositions comprising one or more compounds and one or more pharmaceutically acceptable excipients. A “pharmaceutical composition” refers to a chemical or biological composition suitable for administration to a subject (e.g., mammal). Such compositions can be specifically formulated for administration via one or more of a number of routes, including but not limited to buccal, cutaneous, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. In addition, administration can by means of capsule, drops, foams, gel, gum, injection, liquid, patch, pill, porous pouch, powder, tablet, or other suitable means of administration.

A “pharmaceutical excipient” or a “pharmaceutically acceptable excipient” is a carder, sometimes a liquid, in which an active therapeutic agent is formulated. The excipient generally does not provide any pharmacological activity to the formulation, though it can provide chemical and/or biological stability; and release characteristics. Examples of suitable formulations can be found, for example, in Remington. The Science And Practice of Pharmacy, 20th Edition, (Gennaro, A. R., Chief Editor), Philadelphia College of Pharmacy and Science, 2000, which is incorporated by reference in its entirety.

As used herein “pharmaceutically acceptable carrier” or “excipient” includes, but is not limited to, any and all solvents, dispersion media, coatings, antibacterial and antifungal agents, isotonic and absorption delaying agents that are physiologically compatible. In one embodiment, the carrier is suitable for parenteral administration. Alternatively, the carrier can be suitable for intravenous, intraperitoneal, intramuscular, sublingual, or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art, Except insofar as any conventional media or agent is incompatible with the active compound, use thereof in the pharmaceutical compositions of the invention is contemplated. Supplementary active compounds can also be incorporated into the compositions.

Pharmaceutical compositions can be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants.

In many cases, it will be preferable to include isotonic agents, for example, sugars, polyalcohols such as mannitol, sorbitol, or sodium chloride in the composition. Prolonged absorption of injectable compositions can be brought about by including in the composition an agent which delays absorption, for example, monostearate salts and gelatin. Moreover, the compounds described herein can be formulated in a time release formulation, for example in a composition that includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

Oral forms of administration are also contemplated herein. The pharmaceutical compositions of the present invention can be orally administered as a capsule (hard or soft), tablet (film coated, enteric coated or uncoated), powder or granules (coated or uncoated) or liquid (solution or suspension). The formulations can be conveniently prepared by any of the methods well-known in the art. The pharmaceutical compositions of the present invention can include one or more suitable production aids or excipients including fillers, binders, disintegrants, lubricants, diluents, flow agents, buffering agents, moistening agents, preservatives, colorants, sweeteners, flavors, and pharmaceutically compatible carriers.

For each of the recited embodiments, the compounds can be administered by a variety of dosage forms as known in the art. Any biologically-acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, gum, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradermal), infusions, and combinations thereof.

Other compounds which can be included by admixture are, for example, medically inert ingredients (e.g., solid and liquid diluent), such as lactose, dextrosesaccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal days; thickening agents such as gum tragacanth or sodium alginate, binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients, such as humectants, preservatives, buffers and antioxidants, which are known additives for such formulations.

Liquid dispersions for oral administration can be syrups, emulsions, solutions, or suspensions. The syrups can contain as a carrier, for example, saccharose or saccharose with glycerol and/or mannitol and/or sorbitol. The suspensions and the emulsions can contain a carrier, for example a natural gum, agar, sodium alginate, pectin, methylcellulose, carboxymethylcellulose, or polyvinyl alcohol.

The amount of active compound in a therapeutic composition according to various embodiments of the present invention can vary according to factors such as the disease state, age, gender, weight, patient history, risk factors, predisposition to disease, administration route, pre-existing treatment regime (e.g., possible interactions with other medications), and weight of the subject. Dosage regimens can be adjusted to provide the optimum therapeutic response. For example, a single bolus can be administered, several divided doses can be administered over time, or the dose can be proportionally reduced or increased as indicated by the exigencies of therapeutic situation.

A “dosage unit form,” as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in subjects. In therapeutic use for treatment of conditions in mammals (e.g., humans) for which the compounds of the present invention or an appropriate pharmaceutical composition thereof are effective, the compounds of the present invention can be administered in an effective amount. The dosages as suitable for this invention can be a composition, a pharmaceutical composition or any other compositions described herein.

For each of the recited embodiments, the dosage is typically administered once, twice, or thrice a day, although more frequent dosing intervals are possible. The dosage can be administered every day, every 2 days, every 3 days, every 4 days, every 5 days, every 6 days, and/or every 7 days (once a week). In one embodiment, the dosage can be administered daily for up to and including 30 days, preferably between 7-10 days. in another embodiment, the dosage can be administered twice a day for 10 days. If the patient requires treatment for a chronic disease or condition, the dosage can be administered for as long as signs and/or symptoms persist. The patient can require “maintenance treatment” where the patient is receiving dosages every day for months, years, or the remainder of their lives. In addition, the composition of this invention can be to effect prophylaxis of recurring symptoms. For example, the dosage can be administered once or twice a day to prevent the onset of symptoms in patients at risk, especially for asymptomatic patients.

The absolute weight of a given compound included in a unit dose for administration to a subject can vary widely. For example, about 0.0001 to about 1 g, or about 0.001 to about 0.5 g, of at least one compound of this disclosure, or a plurality of compounds can be administered. Alternatively, the unit dosage can vary from about 0.001 g to about 2 g, from about 0.005 g to about 0.5 g, from about 0.01 g to about 0.25 g, from about 0.02 g to about 0.2 g, from about 0.03 g to about 0.15 g, from about 0.04 g to about 0.12 g, or from about 0.05 g to about 0.1 g.

Daily doses of the compounds can vary as well. Such daily doses can range, for example, from about 0.01 g/day to about 10 g/day, from about 0.02 g/day to about 5 g/day, from about 0.03 g/day to about 4 g/day, from about 0.04 g/day to about 3 g/day, from about 0.05 g/day to about 2 g/day, and from about 0.05 g/day to about 1 g/day.

It will be appreciated that the amount of compound(s) for use in treatment will vary not only with the particular carrier selected but also with the route of administration, the nature of the condition being treated, and the age and condition of the patient. Ultimately the attendant health care provider may determine proper dosage.

The compositions described herein can be administered in any of the following routes: buccal, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. The preferred routes of administration are buccal and oral. The administration can be local, where the composition is administered directly, close to, in the locality, near, at, about, or in the vicinity of, the site(s) of disease, e.g., inflammation, or systemic, wherein the composition is given to the patient and passes through the body widely, thereby reaching the site(s) of disease. Local administration can be administration to, for example, tissue, organ, and/or organ system, which encompasses and/or is affected by the disease, and/or where the disease signs and/or symptoms are active or are likely to occur. Administration can be topical with a local effect, composition is applied directly where its action is desired. Administration can be enteral wherein the desired effect is systemic (non-local), composition is given via the digestive tract. Administration can be parenteral, where the desired effect is systemic, composition is given by other routes than the digestive tract.

The compositions can include the compounds described herein in a “therapeutically effective amount.” Such a therapeutically effective amount is an amount sufficient to obtain the desired physiological effect, such as a reduction of at least one symptom of cancer.

The compositions contemplated herein can contain other ingredients such as chemotherapeutic agents (e.g., abiraterone acetate, alemtuzumab, altretamine, belinostat, bevacizumab, blinatumomab, bleomycin, bortezomib, brentuximab, vedotin, busulfan, cabazitaxel, capecitabine, carboplatin, carmustine, ceritinib, cetuximab, chlorambucil, cisplatin, cladribine, crizotinib, cyclophosphamide, cytarabine, dabrafenib, dacarbazine, dactinomycin dasatinib, daunorubicin, daunoXome, depoCytd docetaxel, doxil I, doxorubicin, epirubicin, eribulin mesylate, erlotinib, estramustine, etoposide, everolimius, floxuridine, fludarabine, fluorouracil, gefitinib, gemcitabine, gliadel wafers, hydroxyurea, ibritumomab, ibrutinib, idarubicin, idelalisib, ifosfamide, imatinib, ipilimumab, irinotecan, ixabepilone, lanreotide, lapatinib, lenalidomide, lenvatinib, lomustine, mechlorethamine, melphalan, mercaptopurine, methotrexate, mitomycin, mitoxantrone, nilotinib, nivolumab, ofatumumab, olaparib, oxaliplatin, paclitaxel, palbociclib, panitumumab, pazopanib, panobinostat, PEG-asparaginase, peginterferon alfa-2b, pembrolizumab, pemetrexed, pentostatin, pralatrexate, procarbazine, ramucirumab, rituximab, romidepsin, ruxolitinib, sipuleucel-T, sorafenib, streptozocin, sunitinib, temozolomide, temsirolimus, teniposide, thalidomide, thioguanine, thiotepa, topotecan, tositumomab, trametinib, trastuzumab, vairubicin, vandetanib, vemurafenib, vinblastine, vincristine, vinorelbine, and the like), anti-inflammatory agents, anti-viral agents, antibacterial agents, antimicrobial agents, immunomodulatory drugs, such as lenalidomide, pomalidomide or thalidomide, histone deacetylase inhibitors, such as panobinostat, preservatives or combinations thereof.

This disclosure also includes methods for treating cancer comprising administering a therapeutically effective amount of at least one of the compounds described herein (e.g., compounds of formulae (I) and (Ia)-(If) to a subject in need thereof. The types of cancers that can be treated include, for example, breast cancer, non-small-cell lung cancer, colorectal cancer, and high-grade gliomas.

As used herein, the terms “treat” and “treating” are not limited to the case where the subject (e.g. patient) is cured and the disease is eradicated. Rather, treatment that merely reduces symptoms, and/or delays disease progression is also contemplated.

The compounds and methods described herein can be used prophylactically or therapeutically. The term “prophylactic” or “therapeutic” treatment refers to administration of a drug to a host before or after onset of a disease or condition. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side, effects therefrom). Administering the compounds described herein (including enantiomers and salts thereof) is contemplated in both a prophylactic treatment (e.g. to patients at risk for disease, such as elderly patients who, because of theft advancing age, are at risk for arthritis, cancer, and the like) and therapeutic treatment (e.g. to patients with symptoms of disease or to patients diagnosed with disease).

The term “therapeutically effective amount” as used herein, refers to that amount of one or more compounds of the various examples of the present invention that elicits a biological or medicinal response in a tissue system, animal or human, that is being sought by a researcher, veterinarian, medical doctor or other clinician, which includes alleviation of the symptoms of the disease or disorder being treated. In some examples, the therapeutically effective amount is that which can treat or alleviate the disease or symptoms of the disease at a reasonable benefit/risk ratio applicable to any medical treatment. However, it is to be understood that the total daily usage of the compounds and compositions described herein can be decided by the attending physician within the scope of sound medical judgment. The specific therapeutically-effective dose level for any particular patient will depend upon a variety of factors, including the condition being treated and the severity of the condition; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician. It is also appreciated that the therapeutically effective amount can be selected with reference to any toxicity, or other undesirable side effect, that might occur during administration of one or more of the compounds described herein.

The term “alkyl” as used herein refers to substituted or unsubstituted straight chain, branched and cyclic, saturated mono- or bi-valent groups having from 1 to 20 carbon atoms, 10 to 20 carbon atoms, 12 to 18 carbon atoms, 6 to about 10 carbon atoms, 1 to 10 carbons atoms, 1 to 8 carbon atoms, 2 to 8 carbon atoms, 3 to 8 carbon atoms, 4 to 8 carbon atoms, 5 to 8 carbon atoms, 1 to 6 carbon atoms, 2 to 6 carbon atoms, 3 to 6 carbon atoms, or 1 to 3 carbon atoms. Examples of straight chain mono-valent (C₁-C₂₀)-alkyl groups include those with from 1 to 8 carbon atoms such as methyl (Le., CH₃), ethyl, n-propyl, n-butyl, n-pentyl, n-hexyl, n-heptyl, n-octyl groups. Examples of branched mono-valent (C₁-C₂₀)-alkyl groups include isopropyl, iso-butyl, sec-butyl, t-butyl, neopentyl, and isopentyl. Examples of straight chain bi-valent (C₁-C₂₀)alkyl groups include those with from 1 to 6 carbon atoms such as —CH₂—, —CH₂CH₂—, —CH₂CH₂CH₂—, —CH₂CH₂CH₂CH₂—, and —CH₂CH₂CH₂CH₂CH₂—. Examples of branched bi-valent alkyl groups include —CH(CH₃)CH₂— and —CH₂CH(CH₃)CH₂—. Examples of cyclic alkyl groups include cyclopropyl, cyclobutyl, cyclopentyl, cyclohexyl, cyclooctyl, bicyclo[1.1.1]pentyl, bicyclo[2.1.1]hexyl, and bicyclo[2.2.1]heptyl. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. In some embodiments, alkyl includes a combination of substituted and unsubstituted alkyl. As an example, alkyl, and also (C₁)alkyl, includes methyl and substituted methyl. As a particular example, (C₁)alkyl includes benzyl. As a further example, alkyl can include methyl and substituted (C₂-C₈)alkyl. Alkyl can also include substituted methyl and unsubstituted (C₂-C₈)alkyl. In some embodiments, alkyl can be methyl and C₂-C₈ linear alkyl. In some embodiments, alkyl can be methyl and C₂-C₈ branched alkyl. The term methyl is understood to be —CH₃, which is not substituted. The term methylene is understood to be —CH₂—, which is not substituted. For comparison, the term (C₁)alkyl is understood to be a substituted or an unsubstituted —CH₃ or a substituted or an unsubstituted —CH₂—. Representative substituted alkyl groups can be substituted one or more times with any of the groups listed herein, for example, cycloalkyl, heterocyclyl, aryl, amino, haloalkyl, hydroxy, cyano, carboxy, nitro, thio, alkoxy, and halogen groups. As further example, representative substituted alkyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. In some embodiments, representative substituted alkyl groups can be substituted from a set of groups including amino, hydroxy, cyano, carboxy, nitro, thio and alkoxy, but not including halogen groups. Thus, in some embodiments alkyl can be substituted with a non-halogen group. For example, representative substituted alkyl groups can be substituted with a fluoro group, substituted with a bromo group, substituted with a halogen other than bromo, or substituted with a halogen other than fluoro. In some embodiments, representative substituted alkyl groups can be substituted with one, two, three or more fluoro groups or they can be substituted with one, two, three or more non-fluoro groups. For example, alkyl can be trifluoromethyl, difluoromethyl, or fluoromethyl, or alkyl can be substituted alkyl other than trifluoromethyl, difluoromethyl or fluoromethyl. Alkyl can be haloalkyl or alkyl can be substituted alkyl other than haloalkyl. The term “alkyl” also generally refers to alkyl groups that can comprise one or more heteroatoms in the carbon chain. Thus, for example, “alkyl” also encompasses groups such as —[(CH₂)ᵣO]ₜH and the like, wherein each r is 1, 2 or 3; and t is 1 to 500.

The term “alkenyl” as used herein refers to substituted or unsubstituted straight chain, branched and cyclic, saturated mono- or bi-valent groups having at least one carbon-carbon double bond and from 2 to 20 carbon atoms, 10 to 20 carbon atoms, 12 to 18 carbon atoms, 6 to about 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. The double bonds can be be trans or cis orientation. The double bonds can be terminal or internal. The alkenyl group can be attached via the portion of the alkenyl group containing the double bond, e.g., vinyl, proper-1-yl and buten-1-yl, or the alkenyl group can be attached via a portion of the alkenyl group that does not contain the double bond, e.g., penten-4-yl. Examples of mono-valent (C₂-C₂₀)-alkenyl groups include those with from 1 to 8 carbon atoms such as vinyl, propenyl, propen-1-yl, propen-2-yl, butenyl, buten-1-yl, buten-2-yl, sec-buten-1-yl, sec-buten-3-yl, pentenyl, hexenyl, heptenyl and octenyl groups. Examples of branched mono-valent (C₂-C₂₀)-alkenyl groups include isopropenyl, iso-butenyl, sec-butenyl, t-butenyl, neopentenyl, and isopentenyl. Examples of straight chain bi-valent (C₂-C₂₀)alkenyl groups include those with from 2 to 6 carbon atoms such as —CHCH—, —CHCHCH₂—, —CHCHCH₂CH₂—, and —CHCHCH₂CH₂CH₂—. Examples of branched bi-valent alkyl groups include —C(CH₃)CH— and —CHC(CH₃)CH₂—. Examples of cyclic alkenyl groups include cyclopentenyl, cyclohexenyl and cyclooctenyl. It is envisaged that alkenyl can also include masked alkenyl groups, precursors of alkenyl groups or other related groups. As such, where alkenyl groups are described it, compounds are also envisaged where a carbon-carbon double bond of an alkenyl is replaced by an epoxide or aziridine ring. Substituted alkenyl also includes alkenyl groups which are substantially tautomeric with a non-alkenyl group. For example, substituted alkenyl can be 2-aminoalkenyl, 2-alkylaminoalkenyl, 2-hydroxyalkenyl, 2-hydroxyvinyl, 2-hydroxypropenyl, but substituted alkenyl is also understood to include the group of substituted alkenyl groups other than alkenyl which are tautomeric with non-alkenyl containing groups. In some embodiments, alkenyl can be understood to include a combination of substituted and unsubstituted alkenyl. For example, alkenyl can be vinyl and substituted vinyl. For example, alkenyl can be vinyl and substituted (C₃-C₈)alkenyl. Alkenyl can also include substituted vinyl and unsubstituted (C₃-C₃)alkenyl. Representative substituted alkenyl groups can be substituted one or more times with any of the groups listed herein, for example, monoalkylamino, dialkylamino, cyano, acetyl, amido, carboxy, nitro, alkylthio, alkoxy, and halogen groups. As further example, representative substituted alkenyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. In some embodiments, representative substituted alkenyl groups can be substituted from a set of groups including monoalkylamino, dialkylamino, cyano, acetyl, amido, carboxy, nitro, alkylthio and alkoxy, but not including halogen groups. Thus, in some embodiments alkenyl can be substituted with a non-halogen group. In some embodiments, representative substituted alkenyl groups can be substituted with a fluoro group, substituted with a bromo group, substituted with a halogen other than bromo, or substituted with a halogen other than fluoro. For example, alkenyl can be 1-fluorovinyl, 2-fluorovinyl, 1,2-difluorovinyl, 1,2,2-trifluorovinyl, 2,2-difluorovinyl, trifluoropropen-2-yl, 3,3,3-trifluoropropenyl, 1-fluoropropenyl, 1-chlorovinyl, 2-chlorovinyl, 1,2-dichlorovinyl, 1,2,2-trichlorovinyl or 2,2-dichlorovinyl. In some embodiments, representative substituted alkenyl groups can be substituted with one, two, three or more fluoro groups or they can be substituted with one, two, three or more non-fluoro groups.

The term “alkynyl” as used herein, refers to substituted or unsubstituted straight and branched chain alkyl groups, except that at least one triple bond exists between two carbon atoms. Thus, alkynyl groups have from 2 to 50 carbon atoms, 2 to 20 carbon atoms, 10 to 20 carbon atoms, 12 to 18 carbon atoms, 6 to about 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. Examples include, but are not limited to ethynyl, propynyl, propyn-1-yl, propyn-2-yl, butynyl, butyn-1-yl, butyn-2-yl, butyn-3-yl, butyn-4-yl, pentynyl, pentyn-1-yl, hexynyl, Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “aryl” as used herein refers to substituted or unsubstituted univalent groups that are derived by removing a hydrogen atom from an arene, which is a cyclic aromatic hydrocarbon, having from 6 to 20 carbon atoms, 10 to 20 carbon atoms, 12 to 20 carbon atoms, 6 to about 10 carbon atoms or 6 to 8 carbon atoms. Examples of (C₆-C₂₀)aryl groups include phenyl, napthalenyl, azulenyl, biphenylyl, indacenyl, fluorenyl, phenanthrenyl, triphenylenyl, pyrenyl, naphthacenyl, chrysenyl, anthracenyl groups. Examples include substituted phenyl, substituted napthalenyl, substituted azulenyl, substituted biphenylyl, substituted indacenyl, substituted fluorenyl, substituted phenanthrenyl, substituted triphenylenyl, substituted pyrenyl, substituted naphthacenyl, substituted chrysenyl, and substituted anthracenyl groups. Examples also include unsubstituted phenyl, unsubstituted napthalenyl, unsubstituted azulenyl, unsubstituted biphenylyl, unsubstituted indacenyl, unsubstituted fluorenyl, unsubstituted phenanthrenyl, unsubstituted triphenylenyl, unsubstituted pyrenyl, unsubstituted naphthacenyl, unsubstituted chrysenyl, and unsubstituted anthracenyl groups. Aryl includes phenyl groups and also non-phenyl aryl groups. From these examples, it is clear that the term (C₆-C₂₀)aryl encompasses mono- and polycyclic (C₆-C₂₀)aryl groups, including fused and non-fused polycyclic (C₆-C₂₀)aryl groups.

The term “heterocyclyl” as used herein refers to substituted aromatic, unsubstituted aromatic, substituted non-aromatic, and unsubstituted non-aromatic rings containing 3 or more atoms in the ring, of which, one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆) or 6 to 8 carbon atoms (C₆-C₈). A heterocyclyl group designated as a C₂-heterocyclyl can be a 5-membered ring with two carbon atoms and three heteroatoms, a 6-membered ring with two carbon atoms and four heteroatoms and so forth. Likewise a C₄-heterocyclyl can be a 5-membered ring with one heteroatom, a 6-membered ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. Representative heterocyclyl groups include, but are not limited to piperidynyl, piperazinyl, morpholinyl, furanyl, pyrrolidinyl, pyridinyl, pyrazinyl, pyrimidinyl, triazinyl, thiophenyl, tetrahydrofuranyl, pyrrolyl, oxazolyl, imidazolyl, triazyolyl, tetrazolyl, benzoxazolinyl, and benzimidazolinyl groups. For example, heterocyclyl groups include, without limitation:

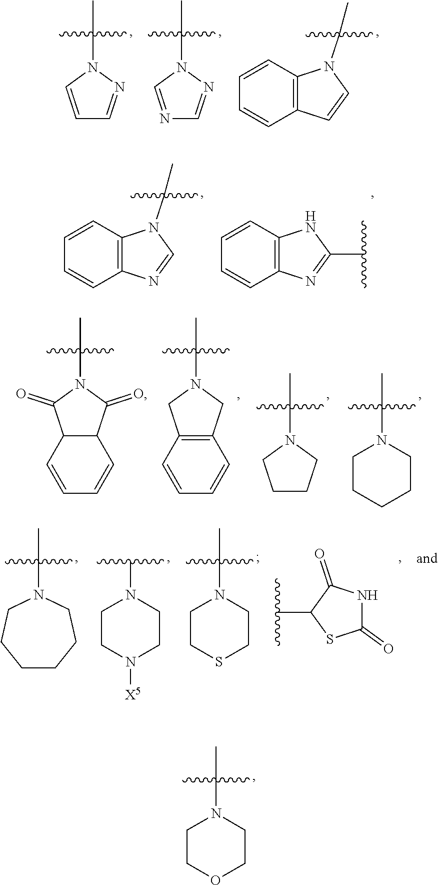

wherein X⁵ represents H, (C₁-C₂₀)alkyl, (C₆-C₂₀)aryl or an amine protecting group (e.g., a t-butyloxycarbonyl group) and wherein the heterocyclyl group can be substituted or unsubstituted. A nitrogen-containing heterocyclyl group is a heterocyclyl group containing a nitrogen atom as an atom in the ring. In some embodiments, the heterocyclyl is other than thiophene or substituted thiophene. In some embodiments, the heterocyclyl is other than furan or substituted furan.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to about 12-20 or about 12-40 carbon atoms bonded to the oxygen atom, and can further include double or triple bonds, and can also include heteroatoms. Thus, alkyoxy also includes an oxygen atom connected to an alkyenyl group and oxygen atom connected to an alkynyl group. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “aryloxy” as used herein refers to an oxygen atom connected to an aryl group as are defined herein.

The term “aralkyl” and “arylalkyl” as used herein refers to alkyl groups as defined herein in which a hydrogen or carbon bond of an alkyl group is replaced with a bond to an aryl group as defined herein. Representative aralkyl groups include benzyl, biphenylmethyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The terms “halo,” “halogen,” or “halide” group, as used herein, by themselves or as part of another substituent, mean, unless otherwise stated, a fluorine, chlorine, bromine, or iodine atom.

The term “amine” and “amino” as used herein refers to a substituent of the form —NH₂, —NHR, —NR², —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, alkenyl, alkynyl, aryl, cycloalkyl, heterocyclyl, group or the like.

The term “formyl” as used herein refers to a group containing a carbonyl moiety wherein the group is bonded via the carbonyl carbon atom. The carbonyl carbon atom is also bonded to a hydrogen atom.

The term “alkoxycarbonyl” as used herein refers to a group containing a carbonyl moiety wherein the group is bonded via the carbonyl carbon atom. The carbonyl carbon atom is also bonded to an oxygen atom which is further bonded to an alkyl group. Alkoxycarbonyl also includes the group where a carbonyl carbon atom is also bonded to an oxygen atom which is further bonded to an alkyenyl group. Alkoxycarbonyl also includes the group where a carbonyl carbon atom is also bonded to an oxygen atom which is further bonded to an alkynyl group. In a further case, which is included in the definition of aikoxycarbonyl as the term is defined herein, and is also included in the term “aryloxycarbonyl,” the carbonyl carbon atom is bonded to an oxygen atom which is bonded to an aryl group instead of an alkyl group.

The term “arylcarbonyl” as used herein refers to a group containing a carbonyl moiety wherein the group is bonded via the carbonyl carbon atom. The carbonyl carbon atom is also bonded to an aryl group.

The term “alkylamido” as used herein refers to a group containing a carbonyl moiety wherein the group is bonded via the carbonyl carbon atom. The carbonyl carbon atom is also bonded to a nitrogen group which is bonded to one or more alkyl groups. In a further case, which is also an alkylamido as the term is defined herein, the carbonyl carbon atom is bonded to a nitrogen atom which is bonded to one or more aryl group instead of, or in addition to, the one or more alkyl group. In a further case, which is also an alkylamido as the term is defined herein, the carbonyl carbon atom is bonded to an nitrogen atom which is bonded to one or more alkenyl group instead of, or in addition to, the one or more alkyl and or/aryl group. In a further case, which is also an alkylamido as the term is defined herein, the carbonyl carbon atom is bonded to a nitrogen atom which is bonded to one or more alkynyl group instead of, or in addition to, the one or more alkyl, alkenyl and/or aryl group.

The term “carboxy” as used herein refers to a group containing a carbonyl moiety wherein the group is bonded via the carbonyl carbon atom. The carbonyl carbon atom is also bonded to a hydroxy group or oxygen anion so as to result in a carboxylic acid or carboxylate. Carboxy also includes both the protonated form of the carboxylic acid and the salt form. For example, carboxy can be understood as COOH or CO₂H.

The term “amido” as used herein refers to a group having the formula C(O)NRR, wherein R is defined herein and can each independently be, e.g., hydrogen, alkyl, aryl or each R, together with the nitrogen atom to which they are attached, form a heterocyclyl group.

The term “alkylthio” as used herein refers to a sulfur atom connected to an alkyl, alkenyl, or alkynyl group as defined herein.

The term “arylthio” as used herein refers to a sulfur atom connected to an aryl group as defined herein.

The term “alkylsulfonyl” as used herein refers to a sulfonyl group connected to an alkyl, alkenyl, or alkynyl group as defined herein.

The term “alkylsulfinyl” as used herein refers to a sulfinyl group connected to an alkyl, alkenyl, or alkynyl group as defined herein.

The term “dialkylaminosulfonyl” as used herein refers to a sulfonyl group connected to a nitrogen further connected to two alkyl groups, as defined herein, and which can optionally be linked together to form a ring with the nitrogen. This term also includes the group where the nitrogen is further connected to one or two alkenyl groups in place of the alkyl groups.

The term “dialkylamino” as used herein refers to an amino group connected to two alkyl groups, as defined herein, and which can optionally be linked together to form a ring with the nitrogen. This term also includes the group where the nitrogen is further connected to one or two alkenyl groups in place of the alkyl groups.

The term “dialkylamido” as used herein refers to an amino group connected to two alkyl groups, as defined herein, and which can optionally be linked together to form a ring with the nitrogen. This term also includes the group where the nitrogen is further connected to one or two alkenyl groups in place of the alkyl groups.

The term “substituted” as used herein refers to a group that is substituted with one or more groups including, but not limited to, the following groups: halogen (e.g., F, Cl, Br, and I), R, OR, ROH (e.g., CH₂OH), OC(O)N(R)₂, ON, NO, NO₂, ONO₂, azido, CF₃, OCF₃, methylenedioxy, ethylenedioxy, (C₃-C₂₀)heteroaryl, N(R)₂, Si(R)₃, SR, SOR, SO₂R, SO₂N(R)₂, SO₃R, P(O)(OR)₂, OP(O)(OR)₂, C(O)R, C(O)C(O)R, C(O)CH₂C(O)R, C(S)R, C(O)OR, OC(O)R, C(O)N(R)₂, C(O)N(R)OH, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(—NH)N(R)₂, C(O)N(OR)R, or C(═NOR)R wherein R can be hydrogen, (C₁-C₂₀)alkyl, (C₆-C₂₀)aryl, heterocyclyl or polyalkylene oxide groups, such as polyalkylene oxide groups of the formula —(CH₂CH₂O)_{f}—R—OR, —(CH₂CH₂CH₂O)_{g}—R—OR, —(CH₂CH₂O)_{f}(CH₂CH₂CH₂O)_{g}—R—OR each of which can, in turn, be substituted or unsubstituted and wherein f and g are each independently an integer from 1 to 50 (e.g., 1 to 10, 1 to 5, 1 to 3 or 2 to 5). Substituted also includes a group that is substituted with one or more groups including, but not limited to, the following groups: fluoro, chloro, bromo, iodo, amino, amino, alkyl, hydroxy, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. Where there are two or more adjacent substituents, the substituents can be linked to form a carbocyclic or heterocyclic ring. Such adjacent groups can have a vicinal or germinal relationship, or they can be adjacent on a ring in, e.g., an ortho-arrangement. Each instance of substituted is understood to be independent. For example, a substituted aryl can be substituted with bromo and a substituted heterocycle on the same compound can be substituted with alkyl. It is envisaged that a substituted group can be substituted with one or more non-fluoro groups. As another example, a substituted group can be substituted with one or more non-cyano groups. As another example, a substituted group can be substituted with one or more groups other than haloalkyl. As yet another example, a substituted group can be substituted with one or more groups other than test-butyl. As yet a further example, a substituted group can be substituted with one or more groups other than trifluoromethyl. As yet even further examples, a substituted group can be substituted with one or more groups other than nitro, other than methyl, other than methoxymethyl, other than dialkylaminosulfonyl, other than bromo, other than chloro, other than amido, other than halo, other than benzodioxepinyl, other than polycyclic heterocyclyl, other than polycyclic substituted aryl, other than methoxycarbonyl, other than alkoxycarbonyl, other than thiophenyl, or other than nitrophenyl, or groups meeting a combination of such descriptions. Further, substituted is also understood to include fluoro, cyano, haloalkyl, tert-butyl, trifluoromethyl, nitro, methyl, methoxymethyl, dialkylaminosulfonyl, bromo, chloro, amido, halo, benzodioxepinyl, polycyclic heterocyclyl, polycyclic substituted aryl, methoxycarbonyl, alkoxycarbonyl, thiophenyl, and nitrophenyl groups.

In some instances, the compounds described herein (e.g., compounds of the formulae (I) and (Ia)-(Id)) can contain chiral centers. AM diastereomers of the compounds described herein are contemplated herein, as well as racemates. Also contemplated herein are isotopomers, which are compounds where one or more atoms in the compound has been replaced with an isotope of that atom. Thus, for example, the disclosure relates to compounds wherein one or more hydrogen atoms is replaced with a deuterium or wherein a fluorine atom is replaced with an ¹⁹F atom.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric (or larger) amount of the appropriate base or acid in water or in an organic solvent, or in a mixture of the two; generally, nonaqueous media like ether, ethyl acetate, ethanol; isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide an active compound, particularly a compound of the invention. Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

As used herein, the term “subject” or “patient” refers to any organism to which a composition described herein can be administered, e.g., for experimental, diagnostic, prophylactic and/or therapeutic purposes. Subject refers to a mammal receiving the compositions disclosed herein or subject to disclosed methods. It is understood and herein contemplated that “mammal” includes but is not limited to humans, nonhuman primates, cows, horses, dogs, cats, mice, rats, rabbits, and guinea pigs.

Each embodiment described above is envisaged to be applicable in each combination with other embodiments described herein. For example, embodiments corresponding to formula (I) are equally envisaged as being applicable to formulae (Ia)-(If).

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed can be resorted to by those of ordinary skill in the art, and that such modifications and variations are considered to be within the scope of embodiments of the present disclosure

The invention is now described with reference to the following Examples. The following working examples therefore, are provided for the purpose of illustration only and specifically point out certain embodiments of the present invention, and are not to be construed as limiting in any way the remainder of the disclosure. Therefore, the examples should be construed to encompass any and all variations which become evident as a result of the teaching provided herein.

One of ordinary skill in the art will recognize that the methods of the current disclosure can be achieved by administration of a composition described herein comprising at least one bronchodilator and at least one pulmonary surfactant via devices not described herein.

Values expressed in a range format should be interpreted in a flexible manner to include not only the numerical values explicitly recited as the limits of the range, but also to include a;; the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, all publications, patents, and patent documents referred to in this document are incorporated by reference herein in theft entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the invention, except when a temporal or operational sequence is explicitly recited. Furthermore, specified steps can be carried out concurrently unless explicit claim language recites that they be carded out separately. For example, a claimed step of doing X and a claimed step of doing Y can be conducted simultaneously within a single operation, and the resulting process will fat within the literal scope of the claimed process.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “substantially no” as used herein refers to less than about 30%, 25%, 20%, 15%, 10%, 5%, 3%, 2%, 1%, 0.5%, 0.1%, 0.05%, 0.001%, or at less than about 0.0005% or less or about 0% or 0%.

Those skilled in the art will appreciate that many modifications to the embodiments described herein are possible without departing from the spirit and scope of the present disclosure. Thus, the description is not intended and should not be construed to be limited to the examples given but should be granted the full breadth of protection afforded by the appended claims and equivalents thereto. In addition, it is possible to use some of the features of the present disclosure without the corresponding use of other features. Accordingly, the foregoing description of or illustrative embodiments is provided for the purpose of illustrating the principles of the present disclosure and not in limitation thereof and can include modification thereto and permutations thereof.

### EXAMPLES

The disclosure can be better understood by reference to the following examples which are offered by way of illustration. The disclosure is not limited to the examples given herein.

### Materials and Methods

All commercially available reagents were used without further purification. Flash column chromatography was performed on a Teledyne-Isco Rf-plus CombiFlash instrument with RediSep columns, NMR spectra were collected on a Bruker Avance III AX-400 or a Bruker Avance III HD-500 equipped with a Prodigy TCI cryoprobe. Chemical shifts (δ) were reported in parts per million (ppm) and referenced to residual solvent signals for Chloroform-d (¹H 7.26 ppm), Dimethyl Sulfoxide-d₆ (¹H 2.50 ppm, ¹³C 39.5 ppm) and Methanol-d₄ (¹H 3.31 ppm, ¹³C 49.0 ppm). Coupling constants (J) are in Hz. Splitting patterns were reported as s (singlet), d (doublet), t (triplet), q (quartet) and m (multiplet). High resolution ESI-MS spectra were recorded on a Thermo Fischer Orbitrap Velos equipped with an autosampler. Where stated, compounds were purified by reverse-phase high-performance liquid chromatography (RP-HPLC) on a C-18 column using 0.1% TFA water and CH₃CN as solvents and TFA salts were quantified using the procedure described by Carlson et. al.⁴⁹

### Purity Analysis

All compounds tested in cells were ≥95% pure by RP-HPLC. Compounds 18-20 were run on a RP-HPLC with a C-18 column over a gradient of 0-10% ACN in 0.1% TFA H₂O over 60 min.

### General Procedure A for the Synthesis of Compounds 1-16, 18-25.

Step 1: The nucleophilic aromatic substitution procedure was adapted from Humphreys et al.³⁵ 4,5-dichloro-2-methylpyridazin-3(2H)-one (1.0 eq.) was stirred in DMSO (1 mL) at room temperature, followed by addition of the primary amine (1.2 eq) and N,N-Diisopropylethylamine (2.0 eq.). The reaction mixture was heated in a sealed tube at 120° C. for 18 h. Following completion of the reaction, the reaction mixture was extracted into ethyl acetate, washed with saturated sodium bicarbonate solution (3×20 mL) and finally with brine (20 mL). The organic layer was dried over magnesium sulfate, filtered, concentrated in vacuo and purified by flash column chromatography (CombiFlash Rf system: 4 g silica, hexanes/ethyl acetate, 0-100% ethyl acetate, 30 minutes unless stated otherwise). The 4- and 5-positional isomers were obtained, with the 5-positional isomer as the more polar fraction. Step 2: The product from Step 1 was stirred in DOM (1 mL) at RT, followed by addition of trifluoroacetic acid (5.0 eq.) and stirred at RT for an additional 2 h. Step 3: The DCM was removed under vacuum and the product was isolated either as a TFA salt or a free base compound. To obtain the TFA salt, cold diethyl ether was added dropwise to precipitate out the product and the diethyl ether was removed in vacuo. For the free amine compounds, the mixture from Step 2 was extracted into DCM and treated with 1 M NaOH to attain a pH>10. The DCM layer was dried with magnesium sulfate, filtered and the DCM was removed in vacua to obtain the product.

### General Procedure B for the Synthesis of Compounds 26-28.

4,5-dichloropyridazin-3(2H)-one (1.0 eq.) was stirred in DMF (5 mL) followed by addition of sodium hydride (1.1 eq) and the alkyl bromide (1.4 eq.). The reaction mixture was stirred at room temperature for 12 h. Following completion of the reaction, the reaction mixture was extracted into ethyl acetate, washed with distilled water and finally with brine. The organic layer was dried over magnesium sulfate, filtered, concentrated in vacua and purified by flash column chromatography (CombiFlash Rf system: 24 g silica, hexanes/ethyl acetate, 0-100% ethyl acetate, 20 minutes).

GSK4027 was purchased from Cayman Chemicals and has the formula:

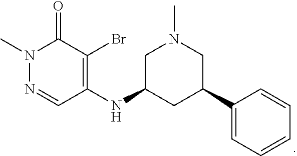 [SMILES: CN1C[C@H](Nc2cnn(C)c(=O)c2Br)C[C@H](c2ccccc2)C1]

The synthesis and characterization of compounds 1-3 were described previously.

Protein-Observed Fluorine (PrOF) NMR. Fluorinated BPTF, PCAF, CECR2 and BRD4 D1 were expressed and purified as described previously. 40-50 μM of protein in 50 mM TRIS, 100 mM NaCl, and pH 7.4 was diluted by adding 25 μL of D₂O and 2 μL of 0.1% TFA for NMR locking and referencing purposes, respectively. Two spectra were taken of the control protein sample in the presence of 5 μL of DMSO (1% final concentration) at an O1P=−75 ppm, NS=16, d1=1 s, AQ=0.5 s (samples were referenced to trifluoroacetate at −75.25 ppm) and an O1P=−125 ppm, NS=500-750, d1=0.7 s, AQ=0.05 s (protein resonances). Ligands were titrated and the change in chemical shift relative to the control sample was plotted as a function of ligand concentration to generate binding isotherms. The data was processed in Mestrenova and isotherms were fit using GraphPad Prism with the equation below. Δδ_{obs} is the change in chemical shift, [L] is the total ligand concentration, and [P] is the total protein concentration:

$${Δδ}_{obs} = {Δδ}_{max} \frac{( {K}_{d} + [ L ] + [ P ] ) - \sqrt{{( {K}_{d} + [ L ] + [ P ] )}^{2} - 4 [ PL ]}}{2 [ PL ]}$$

General Procedure for AlphaScreen Assay.³⁶ Unlabeled His₉-tagged BPTF and BRD4 D1 were expressed and purified as described previously.³⁶ The AlphaScreen assay procedures for BPTF and BRD4 bromodomains were adapted from the manufacturers protocol (PerkinElmer, USA). Nickel chelate (Ni-NTA) acceptor beads and streptavidin donor beads were purchased from PerkinElmer (Cat. #: 6760619M). The biotinylated Histone H4 KAc5,8,12,16 peptide was purchased from EpiCypher, with the sequence:

(SEQ ID NO: 1)
Ac-SGRGK(Ac)GGK(Ac)GLGK(Ac)GGAK(Ac)RHRKVLR-
Peg(Biot).

All reagents were diluted in the assay buffer (50 mM HEPES-Na⁺ (ChemImpex), 100 mM NaCl (SigmaAldrich), 0.05% CHAPS (RPI), 0.1% BSA (SigmaAldrich), pH 7.4). The final assay concentrations (after the addition of all assay components) of 30 nM for His₉-tagged BPTF bromodomain and 50 nM for the biotinylated peptide were used. For BRD4 D1, 7.5 nM His₉-BRD4 and 25 nM of the peptide were used. 3-fold serial dilutions were prepared with varying concentrations of the compounds and a fixed protein concentration, keeping the final DMSO concentration at either 0.25% or 0.5% v/v, depending upon the solubility of the compounds. 5 μL of these solutions were added to a 384-well plate (ProxiPlate-384, Perkin Elmer). The plate was sealed and kept at room temperature for 30 min, followed by the addition of 5 μL of the biotinylated peptide. 5 μL of nickel chelate acceptor beads was added to each well under low light conditions (<100 lux), to a final concentration of 20 μg/mL, and the plate was incubated at room temperature in the dark for 30 minutes. This was followed by the addition of 5 μL (20 μg/mL final concentration) of streptavidin donor beads in low light conditions. After incubation for 30 min in the dark, the plate was read in AlphaScreen mode using a PerkinElmer EnSpire plate reader. Each compound was run in two technical replicates and the data was normalized against 0 μM inhibitor signal to obtain the % normalized AlphaScreen signal. IC₅₀ values were calculated in GraphPad Prism 5 using sigmoidal 4-parameter logistic (4PL) curve

Cell culture methods. 4T1 cells were grown to a confluency of 50-60% using media containing DMEM with 10% fetal bovine serum (FES), 2 mM glutamine and penicillin-streptomycin. 4T1 cells with snRNA-mediated BPTF knockdown (KD) were prepared as described previously. For NURF inhibitor toxicity study, 4000 cells/well were seeded in a 96-well plate and allowed to adhere overnight. The next day, 10 different dilutions of inhibitors were prepared starting with a highest concentration of 1.0 mM and further serially diluted 10 times to get the lowest concentration of 1.95 μM. Cells were treated with the inhibitors in complete media for 4 days. Thereafter, the MTS reagent was prepared using the CellTitre 96 aqueous MTS reagent (Promega, Cat #G1111) and phenazine methosulphate (Sigma, Cat #P9625). The MTS assay was performed as per manufacturer's protocol and the absorbance was recorded at a wavelength of 490 nm. Fraction cell survival was calculated using untreated control cells to indicate complete survival (1.0) and blank solutions as 0.0 survival. The data was derived from three independent experiments (N=3) and fraction survival was plotted as mean fraction survival±SEM using GraphPad Prism software. For checking the toxicity on wildtype and BPTF knockdown cells, three doses were selected for each inhibitor based on theft toxicity curves and treated for 4 days alone or in combination with 50 nM doxorubicin. Fraction survival was measured and calculated by MIS assay as mentioned above.

Cytotoxicity experiments with Eph4 cells. Eph4 cells were treated with either DMSO, AU1, 19 or 20 for 72 hours. Media containing each condition were changed every 12 hours. Cells were then incubated with Magic Red Caspase 3/7 (ImmunoChemistry Technologies, #936) to manufacturers specifications. Cells were also stained with Live/Dead Violet (Thermo Scientific, #L34964) in accordance to manufacturers specifications. All flow was performed on a Macsquant 10 (Miltenyl Biotec) and analyzed on FlowJo (TreeStar/BD). Statistically significant differences for cell line treatment groups were considered with a t-test p-value lower than 0.05 (p<0.05).

qPCR methods. Eph4 cells were treated for 24 h and harvested in trizol. RNA extraction was carried out via chloroform extractions. cDNA creation was completed via SuperScript III cDNA creation kit (Invitrogen, #12574026). All qpcrs are normalized to EPH4 DMSO and the house keeping gene beta actin. Bars represent 2 biological replicates and 3 technical replicates. All statistical analysis are student's t-test carried out on GraphPad. Reactions were carried out on the Quantstudio 6 platform using Sybr Green PCR Master Mix (Applied Biosystems, #4309155) Statistically significant differences for cell line treatment groups were considered with a one-way Anova p-value lower than 0.05 (p<0.05).

UV-Vis Methods. Compounds were diluted in DMSO at a top concentration of 100 mM. 2-fold serial dilutions in DMSO where performed followed by 1000-fold dilution into phosphate saline buffer (PBS) to get a final top concentration of 100 μM in 0.1% DMSO for each compound. UV-Vis measurements at 254 nm were taken on a Biomate 3S Spectrophotometer.

X-ray crystallography conditions and data collection methods. BPTF bromodomain purification and crystallography for compounds 1-4: Protein purification was performed at 4° C. by FPLC using columns and chromatography resins from GE Healthcare. Cell pellets were re--suspended in 50 mM Na/K Phosphate buffer (pH 7.4) containing 100 mM NaCl, 20 mM imidazole, 0.01% w/v lysozyme, 0.01% v/v Triton X-100 and 1mM DTT. Cells were lysed using a homogenizer, the lysate was clarified by centrifugation and subjected to purification on immobilized Ni²⁺-affinity chromatography (Qiagen) using a linear gradient of 20-500 mM imidazole. Fractions containing BPTF were pooled and incubated overnight with TEV protease at 4° C. Cleaved BPTF was subjected to a second Ni²⁺-affinity chromatography run to remove His-TEV and the cleaved His-tag. The flow-through containing BPTF was concentrated and purified to homogeneity by size exclusion chromatography using a Superdex 26/60 column. Protein was eluted using 50 mM Tris/HCl (pH 8.0) containing 100 mM NaCl and 1 mM DTT. Peak fractions were combined, concentrated to 5 mg/mL, flash frozen in liquid N₂ and stored at −80 ° C. Crystallization was performed at 18° C. with precipitant solutions from Hampton Research using a Mosquito liquid handler (TTP Labtech). Robust crystallization conditions were established using 25% PEG 3,350, 0.2 M lithium sulfate monohydrate, 0.1 M Bis-Tris pH 6.5 mixed with an equal volume of protein in vapor diffusion hanging droplets. Compounds were cocrystallized with BPTF at 1 mM final concentration. Crystals were cryoprotected by addition of 20% ethylene glycol in the precipitant, flash frozen and stored in liquid N₂. During data collection, crystals were maintained under a constant stream of N₂ gas. X-ray diffraction data were recorded at beamlines 22-BM hosted by Ser-Cat and 23-ID-D hosted by GM/CA of Argonne National Laboratory. Data were indexed and scaled with XDS.⁵⁰ Phasing and refinement was performed using PHENIX⁵¹ and model budding with Coot.⁵² PDB entry 7K6R served as the search model for molecular replacement. Initial models for small molecule ligands were generated through MarvinSketch (ChemAxon, Cambridge, MA) and ligands restraints through eLBOW of the PHENIX suite. All structures have been validated by MolProbity. Figures were prepared using PyMOL (Schrodinger, LLC). Data processing and refinement statistics are given in Table S2.

Crystallography methods for compounds 10-13: Unlabeled BPTF was expressed and purified as described previously.³⁶ 200-300 μM BPTF (in 50 mM TRIS, 100 mM NaCl, 10% (v/v) ethylene glycol, pH 7.4) was crystallized with 700 μM of compounds 10-13 using the hanging drop method at 4° C. Crystals grew to harvestable size in 3-4 days. 10 was crystallized using 200 mM potassium acetate and 20% (v/v) PEG 3350. 11 and 13 were crystallized using 200 mM manganese acetate and 20% (v/v) PEG 3350. 12 was crystallized with 200 mM magnesium chloride and 10% (v/v) PEG 3350. Crystals were harvested, cryoprotected with ethylene glycol and flash frozen. Data was acquired at the Advanced Photon Source with the NECAT 24-IDE beamline. The structures were solved using molecular replacement with Phaser-MR and the PDB structure 3UV2. PHENIX⁵¹ and Coot⁵² were used for structure refinement and model building. Data processing and refinement statistics are given in Table S3.

Crystallography method for compound 19: Unlabeled BPTF was expressed and purified as described previously.³⁶ RPTF was concentrated to 16 mg/mL and previously reported crystallization conditions⁵³ were chosen for optimization using a Dragonfly liquid handler (TTP Labtech). Drops consisting of 150 nL reservoir solution and 150 nL protein solution were set up in 96-well hanging drop plates using a mosquito crystallization robot (TTP Labtech). Thin needles formed and grew over 14-16 days in 0.2M NaCl and 23% PEG 3350 at 277 K. Larger needle crystals were grown in 24-well VDX hanging drop plate using micro-seeding. These crystals were soaked in solutions containing 1 mM of compound 19 for 1 hour, cryo-protected using the well solution supplemented with additional 10% glycerol, flash frozen and X-ray diffraction data were collected at 100 K on beam line SER-CAT 221D at the Advanced Photon Source. Diffraction images were indexed, integrated, and scaled using HKL2000 suite. Phases were obtained by rigid body refinement using 3UV2 as the initial model. Residues were renumbered using 7K6R as a template. Model building was carried out using Coot. The final model was refined using PHENIX, and torsion-angle molecular dynamics with a slow-cooling simulated annealing. Data processing and refinement statistics are given in Table S4.

### Example 1

5-(azetidin-3-ylamino)-4-chloro-2-methylpyridazin-3(2H)-one (4). Following the general procedure A, (4,5-dichloro-2-methylpyridazin-3(2H)-one (150 mg, 0.838 mmol, 1.0 eq.), tert-butyl 3-aminoazetidine-1-carboxylate (173 mg, 1.01 mmol, 1.2 eq.), N,N-Diisopropylethylamine (292 μL, 1.68 mmol, 2.0 eq.)), product 4 was obtained as a brown solid (211 mg, 77% yield over two steps). ¹H NMR (400 MHz, DMSO-d₆) δ 8.78 (d, J=68.6 Hz, 2H), 7.76 (s, 1H), 7.16 (d, J=7.3 Hz, 1H), 4.76 (h, J=7.5 Hz, 1H), 4.28-4.19 (m, 2H), 4.18-4.08 (m, 2H), 3.60 (s, 3H). ¹³C NMR (126 MHz, DMSO-d₆) δ 158.5 (q, J=34.9 Hz), 156.8, 143.2, 126.6, 116.1 (q, J=293.4 Hz), 106.4, 52.6, 44.1 (one resonance obscured by solvent). HRMS (ESI-TOF) calculated for C₈H₁₂ClN₄O⁺ [M+H]⁺: 215.0694, observed 215.0686.

### Example 2

5-(azepan-3-ylamino)-4-chloro-2-methylpyridazin-3(2H)-one (5). Following the general procedure A, (4,5-dichloro-2-methylpyridazin-3(2H)-one (114 mg, 0.636 mmol, 1.0 eq.), tert-butyl 3-aminoazepane-1-carboxylate (150 mg, 0.699 mmol, 1.1 eq.), N,N-Diisopropylethylamine (222 μL, 1.27 mmol, 2.0 eq.)), product 5 was obtained as a brown oil (49 mg, 21% yield over two steps). ¹H NMR (500 MHz, DMSO-d₆) δ 9.05 (s, 2H), 7.93 (s, 1H), 6.35 (d, J=9.2 Hz, 1H), 4.27-4.14 (m, 1H), 3.60 (s, 3H), 3.27-3.09 (m, 4H), 2.06-1.94 (m, 1H), 1.91-1.83 (m, 1H), 1.82-1.71 (m, 3H), 1.62-1.47 (m, 1H), ¹³C NMR (126 MHz, DMSO-d₆) δ 158.5, 156.8, 143.2, 126.5, 105.7, 49.4, 49.4, 46.4, 33.2, 24.8, 22.1 (one resonance obscured by solvent). HRMS (ESI-TOF) calculated for C₁₁H₁₈ClN₄O⁺ [M+H]⁺: 257.1164, observed 257.1154.

### Example 3

4-chloro-2-methyl-5-(phenylamino)pyridazin-3(2H)-one (6). Following step 1 of the general procedure A, (4,5-dichloro-2-methylpyridazin-3(2H)-one (634 mg, 3.54 mmol, 1.1 eq.), aniline (300 mg, 3.22 mmol, 1.0 eq.), N,N-Diisopropylethylamine (1.12 mL, 6.44 mmol, 2.0 eq.)), product 6 was obtained as a yellow solid (73 mg, 10% yield over two steps). ¹H NMR (500 MHz, DMSO-d₆) δ 8.73 (s, 1H), 7.64 (d, J=1.7 Hz, 1H), 7.39 (d, J=7.7 Hz, 2H), 7.25 (d, J=7.9 Hz, 2H), 7.20 (t, J=7.4 Hz, 1H), 3.61 (s, 3H). ¹³C NMR (126 MHz, DMSO-d₆) δ 157.5, 142.8, 139.0, 129.9, 128.1, 125.4, 124.0, 109.0 (one resonance obscured by solvent). HRMS (ESI-TOF) calculated for C₁₁H₁₁ClN₃O⁺ [M+H]+: 236.0585, observed 236.0575.

### Example 4

5-((4-aminophenyl)amino)-4-chloro-2-methylpyridazin-3(2H)-one (7). Following the general procedure A, (4,5-dichloro-2-methylpyridazin-3(2H)-one (300 mg, 1.68 mmol, 1.0 eq.), tert-butyl (4-aminophenyl)carbamate (419 mg, 2.01 mmol, 1.2 eq.), N,N-Diisopropylethylamine (584 μL, 3.35 mmol, 2.0 eq.)), product 7 was obtained as a brown solid (49 mg, 8% yield over two steps). ¹H NMR (500 MHz, Methanol-d₄) δ 7.49 (s, 1H), 7.00 (d, J=8.6 Hz, 2H), 6.77 (d, J=8.6 Hz, 2H), 4.59 (s, 1H), 3.70 (s, 3H). ¹³C NMR (126 MHz, Methanol-d₄) δ 160.1, 148.3, 146.2, 129.2, 128,8, 128.3, 117.0, 107.4, 40.5. HRMS (ESI-TOF) calculated for C₁₁H₁₂ClN₄O⁺ [M+H]⁺: 251.0694, observed 251.0683.

### Example 5

4-chloro-5-((4-fluorophenyl)amino)-2-methylpyridazin-3(2H)-one (8). Following step 1 of the general procedure A, (4,5-dichloro-2-methylpyridazin-3(2H)-one (532 mg, 2.97 mmol, 1.1 eq.), 4-fluoroaniline (300 mg, 2.70 mmol, 1.0 eq.), N,N-Diisopropylethylamine (940 5.40 mmol, 2.0 eq.)), product 8 was obtained as a white solid (52 mg, 8% yield over two steps). ¹H NMR (500 MHz, DMSO-d₆) δ 8.69 (s, 1H), 7.57 (s, 1H), 7.34-7.18 (m, 4H), 3.60 (s, 3H). ¹³C NMR (126 MHz, DMSO-d₆) δ ¹³C NMR (126 MHz, DMSO-d₆) δ 159.5 (d, J=242.0 Hz), 157.0, 142.6, 134.8, 134.7, 127.4, 126.2 (d, J=8.5 Hz), 116.1 (d, J =22.6 Hz), 108.0 (one resonance obscured by solvent). HRMS (ESI-TOF) calculated for C₁₁H₁₀ClFN₃O⁺ [M+H]⁺: 254.0491, observed 254.0477.

### Example 6

5-((3-aminobenzyl)amino)-4-chloro-2-methylpyridazin-3(2H)-one (9). Following the general procedure A, (4,5-dichloro-2-methylpyridazin-3(2H)-one (150 mg, 0.838 mmol, 1.0 eq.), tert-butyl (3-(aminomethyl)phenyl)carbamate (224 mg, 1.01 mmol, 1.2 eq.), N,N-Disopropylethylamine (292 μL, 1.68 mmol, 2.0 eq.)), product 9 was obtained as a brown solid (50 mg, 23% yield over two steps). ¹H NMR (500 MHz, DMSO-d₆) δ 7.61 (s, 1H), 7.22 (t, J=6.5 Hz, 1H), 6.96 (t, J=7.7 Hz, 1H), 6.45 (d, 1.9 Hz, 1H), 6.42 (dt, J=7.9, 1.9 Hz, 2H), 5.08 (s, 2H), 4.41 (d, J=6.5 Hz, 2H), 3.54 (s, 3H). ¹³C NMR (126 MHz, DMSO-d₆) δ 156.8, 149.0, 144.7, 139.6, 129.1, 126.5, 113.9, 112.7, 111.6, 104.8, 45.3 (one resonance obscured by solvent). HRMS (ESI-TOF) calculated for C₁₂H₁₄ClN₄O⁺ [M+H]⁺: 265.0851, observed 265.0842.

### Example 7

5-((4-(aminomethyl)phenyl)amino)-4-chloro-2-methylpyridazin-3(2H)-one (10). Following the general procedure A, (4,5-dichloro-2-methylpyridazin-3(2H)-one (250 mg, 1.39 mmol, 1.0 eq.), tert-butyl (4-aminobenzyl)carbamate (279 mg, 1.26 mmol, 0.9 eq.), N,N-Diisopropylethylamine (487 μL, 2.79 mmol, 2.0 eq.)), product 10 was obtained as a brown solid (65 mg, 14% yield over two steps). ¹H NMR (500 MHz, DMSO-d₆) δ 8.84 (s, 1H), 8.13 (s, 3H), 7.63 (s, 1H), 7.46 (d, J=8.2 Hz, 2H), 7.29 (d, J=8.5 Hz, 2H), 4.03 (q, J=5.6 Hz, 2H), 3.62 (s, 3H). ¹³C NMR (126 MHz, DMSO-d₆) δ 157.1, 142.1, 138.9, 130.2, 130.1, 127.8, 123.1, 109.3, 41.85 (one resonance obscured by solvent). HRMS (ESI-TOF) calculated for C₁₂H₁₄ClN₄O⁺ [M+H]⁺: 265.0851, observed 265.0839.

### Example 8

4-chloro-5-((4-((dimethylamino)methyl)phenyl)amino)-2-methylpyridazin-3(2H)-one (11). Following step 1 of the general procedure A, 4,5-dichloro-2-methylpyridazin-3(2H)-one (328 mg, 1.83 mmol, 1.1 eq.), 4-((dimethylamino)methyl)aniline (245 μL, 1.66 mmol, 1.0 eq.), N,N-Diisopropylethylamine (579 μL, 3.32 mmol, 2.0 eq.) and purification by flash column chromatography (CombiFlash Rf system: 4 g silica, DCM/methanol, 0-20% methanol, 20 minutes), product 11 was obtained as a brown solid (46 mg, 9% yield), ¹H NMR (500 MHz, Chloroform-d) δ 7.66 (s, 1H), 7.36 (d, J=7.9 Hz, 2H), 7.15 (d, J=7.9 Hz, 2H), 6.39 (s, 1H), 3.76 (s, 3H), 3.43 (s, 2H), 2.26 (s, 6H) (NH not observed). ¹³C NMR (126 MHz, DMSO-d₆) δ 157.0, 142.4, 137.2, 135.7, 129.7, 127.6, 123.5, 108.2, 62.8, 44.9. HRMS (ESI-TOF) calculated for C₁₄₁₈ClN₄O⁺ [M+H]⁺: 293.1164, observed 293.1150.

### Example 9

4-chloro-2-methyl-5-((1,2,3,4-tetrahydroisoquinol-6-yl)amino)pyridazin-3(2H)-one (12). Following the general procedure A, (4,5-dichloro-2-methylpyridazin-3(2H)-one (198 mg, 1.11 mmol, 1.1 eq.), tert-butyl 6-amino-3,4-dihydroisoquinoline-2(1H)-carboxylate (250 mg, 1.01 mmol, 1.0 eq.), N,N-Diisopropylethylamine (351 μL, 2.01 mmol, 2.0 eq.)), product 12 was obtained as a yellow solid (51 mg, 17% yield over two steps). ¹H NMR (500 MHz, DMSO-d₆) δ 8.62 (s, 1H), 7.60 (s, 1H), 7.05 (d, J=8.2 Hz, 1H), 6.99 (dd, J=8.3, 2.2 Hz, 1H), 6.96 (s, 1H), 3.85 (s, 2H), 3.60 (s, 3H), 2.96 (t, J=5.7 Hz, 2H), 2.69 (t, J=6.0 Hz, 2H). ¹³C NMR (126 MHz, DMSO-d₆) δ 157.0, 142.6, 136.0 (two resonances partially overlapping), 132.8, 127.5, 127.1, 124.2, 121.4, 107.8, 47.1, 42.8, 28.4. HRMS (ESI-TOF) calculated for C₁₄H₁₆ClN₄O⁺ [M+H]⁺: 291.1007, observed 291.0996.

### Example 10

4-chloro-2-methyl-5-((1,2,3,4-tetrahydroisoquinol in-7-yl)amino)pyridazin-3(2H)-one (13). Following the general procedure A, (4,5-dichloro-2-methylpyridazin-3(2H)-one (198 mg, 1.11 mmol, 1.1 eq.), tert-butyl 7-amino-3,4-dihydroisoquinoline-2(1H)-carboxylate (250 mg, 1.01 mmol, 1.0 eq.), N,N-Diisopropylethylamine (351 μL, 2.01 mmol, 2.0 eq.)), product 13 was obtained as a yellow solid (29 mg, 10% yield over two steps). ¹H NMR (500 MHz, DMSO-d₆) δ 8.61 (s, 1H), 7.59 (s, 1H), 7.09 (d, J=8.1 Hz, 1H), 6.99 (dd, J=8.1, 2.2 Hz, 1H), 6.90 (d, J=2.3 Hz, 1H), 3.83 (s, 2H), 3.60 (s, 3H), 2.95 (t, J=5,9 Hz, 2H), 2.67 (t, J=5.9 Hz, 2H) (NH not observed). ¹³C NMR (126 MHz, DMSO-d₆) δ 157.0, 142.6, 136.9, 135.7, 131.9, 129.9, 127.5, 121.7, 121.5, 107.8, 47.3, 43.0, 27.9. HRMS (ESI-TOF) calculated for C₁₄H₁₆ClN₄O⁺ [M+H]⁺: 291.1007, observed 291.0995.

### Example 11

4-chloro-2-methyl-5-((5,6,7,8-tetrahydronaphthalen-2-yl)amino)pyridazin-3(2H)-one (14). Following step 1 of the general procedure A, (4,5-dichloro-2-methylpyridazin-3(2H)-one (334 mg, 1.87 mmol, 1,1 eq.), 5,6,7,8-tetrahydronaphthalen-2-amine (250 mg, 1.70 mmol, 1.0 eq.), N,N-Diisopropylethylamine (592 μL, 3.40 mmol, 2.0 eq.)), product 14 was obtained as a yellow solid (175 mg, 36% yield over two steps). ¹H NMR (500 MHz, DMSO-d₆) δ 8.57 (s, 1H), 7.59 (s, 1H), 7.06 (d, J=8.1 Hz, 1H), 6.97-6.92 (m, 2H), 3.59 (s, 3H), 2.70 (t, J=4.8, 2.4 Hz, 4H), 1.72 (t, J=3.3 Hz, 4H), ¹³C NMR (126 MHz, DMSO) δ 157.5, 143.1, 138.2, 136.1, 134.2, 130.2, 127.9, 124.7, 121.8, 108.1, 29.2, 28.8, 23.2, 23.0 (one resonance obscured by solvent). HRMS (ESI-TOF) calculated for C₁₅H₁₇ClN₃O⁺ [M+H]⁺: 290.1055, observed 290.1038.

### Example 12

5-((3-(aminomethyl)phenyl)amino)-4-chloro-2-methylpyridazin-3(2H)-one (15). Following the general procedure A, (4,5-dichloro-2-methylpyridazin-3(2H)-one (250 mg, 1.39 mmol, 1.0 eq.), tert-butyl (3-aminobenzyl)carbamate (279 mg, 1.26 mmol, 0.9 eq.), N,N-Diisopropylethylamine (487 μL, 2.79 mmol, 2.0 eq.)), a portion of the crude product (71 mg, crude 15% yield) was then purified by reverse-phase HPLC (5-40% CH₃CN gradient over 30 minutes) to obtain product 15 as a brown solid. ¹H NMR (500 MHz, DMSO-d₆) δ 8.84 (s, 1H), 8.14 (s, 3H), 7.75 (s, 1H), 7.44 (m, 1H), 7.31 (m, 1H), 7.25 (m, 2H), 4.03 (q, J=5.7 Hz, 2H), 3.63 (s, 3H), ¹³C NMR (126 MHz, DMSO-d₆) δ 157.0, 142.0, 138.9, 135.2, 129.8, 127.8, 125.1, 123.4, 123.0, 109.3, 42.1 (one resonance obscured by solvent). HRMS (ESI-TOF) calculated for C₁₂H₁₄ClN₄O⁺ [M+H]⁺: 265.0851, observed 265.0842.

### Example 13

5-((2-(aminomethyl)phenyl)amino)-4-chloro-2-methylpyridazin-3(2H)-one (16). Compound previously characterized in literature.³⁵ Following the general procedure A, (4,5-dichloro-2-methylpyridazin-3(2H)-one (250 mg, 1.39 mmol, 1.0 eq.), tert-butyl (2-aminobenzyl)carbamate (279 mg, 1.26 mmol, 0.9 eq.), N,N-Diisopropylethylamine (487 μL, 2.79 mmol, 2.0 eq.)), a portion of the crude product (12 mg, 3% yield) was then purified by reverse-phase HPLC (5-40% CH₃CN gradient over 30 minutes) to obtain product 16 as a brown solid. ¹H NMR (500 MHz, DMSO-d₆) δ 8.34 (s, 1H), 8.12 (s, 3H), 7.50 (m, 1H), 7.36 (m, 2H), 7.22 (m, 1H), 7.26 (s, 1H), 4.06 (q, J=5.2 Hz, 2H), 3.61 (s, 3H). HRMS (ESI-TOF) calculated for C₁₂H₁₄ClN₄O⁺ [M+H]⁺: 265.0851, observed 265.0840.

### Example 14

N-(4-((5-chloro-1-methyl-6-oxo-1,6-dihydropyridazin-4-yl)amino)benzyl)acetamide (17). 5-((4-(aminomethyl)phenyl)amino)-4-chloro-2-methylpyridazin-3(2H)-one (10) (20 mg, 0.05 mmol, 1.0 eq.) was stirred in dichloromethane (0.5 mL) followed by addition of acetic anhydride (6.4 mg, 0.06 mmol, 1.2 eq) and triethylamine (26 mg, 0.26 mmol, 5 eq.). The reaction mixture was stirred at room temperature for 0.5 h. Following completion of reaction, the reaction mixture was washed with diethyl ether. The solid was concentrated in vacuo and purified by flash column chromatography (CombiFlash Rf system: 12 g silica, dichloromethane/methanol, 0-10% methanol, 20 minutes). Product 17 was obtained as a white solid (9 mg, 56% yield). ¹H NMR (500 MHz, Chloroform-d) δ 7.66 (s, 1H), 7.35 (d, J=8.4 Hz, 2H), 7.16 (d, J=8.3 Hz, 2H), 4.46 (d, J=8.3 Hz, 2H), 3.77 (s, 3H), 2.08 (s, 3H). ¹³C NMR (126 MHz, Chloroform-d) δ 170.3, 157.9, 142.1, 136.8, 136.7, 129.4, 126.7, 124.2, 110.2, 43.2, 40.4, 23.4. HRMS (ESI-TOF) calculated for C₁₄H₁₆ClN₄O₂^{′} [M+H]⁺: 307.0956, observed 307.0949.

### Example 15

5-((4-(2-aminoethyl)phenyl)amino)-4-chloro-2-methylpyridazin-3(2H)-one (18/BZ1). Following the general procedure A, (4,5-dichloro-2-methylpyridazin-3(2H)-one (250 mg, 1.39 mmol, 1.0 eq.), tert-butyl (2-(4-amino-phenyl)-ethyl)carbamate (307 mg, 1.30 mmol, 0.9 eq.), N,N-Diisopropylethylamine (487 μL, 2.79 mmol, 2.0 eq.)), product 18 was obtained as a brown solid (140 mg, 21% yield over two steps). ¹H NMR (500 MHz, DMSO-d₆) δ 8.69 (s, 1 H), 7.61 (s, 1 H), 7.27 (d, 8.0 Hz, 2H), 7.20 (d, J=7.9 Hz, 2H), 3.61 (s, 3 H), 2.93 (t, J=7.5 Hz, 2 H), 2.76 (t, J=7.6 Hz, 2H). ¹³C NMR (126 MHz, DMSO-d₆) δ 157.1, 142,6, 136.7, 135.6, 129.7, 127.6, 123.9, 108.1, 41.5, 35.4 (one resonance obscured by solvent). HRMS (ESI-TOF) calculated for C₁₃H₁₆ClN₄O⁺ [M+H]⁺: 279.1007, observed 279.1002.

### Example 16

4-chloro-5-((4-(2-(dimethylamino)ethyl)phenyl)amino)-2-methylpyridazin-3(2H)-one (19). Following step 1 of the general procedure A, (4,5-dichloro-2-methylpyridazin-3(2H)-one (250 mg, 1.39 mmol, 1.0 eq.), 4-(2-dimethylamino -ethyl)aniline (214 mg, 1.26 mmol, 0.9 eq.), N,N-Diisopropylethylamine (487 μL, 2.79 mmol, 2.0 eq.)) and purification by flash column chromatography (CombiFlash Rf system: 4 g silica, DCM/methanol, 0-20% methanol, 20 minutes), product 19 (more polar fraction) was obtained as a yellow solid (42 mg, 11% yield). ¹H NMR (500 MHz, DMSO-d₆) δ 8.65 (s, 1H), 7.59 (s, 1H), 7.25 (d, J=8.3 Hz, 2H), 7.16 (d, J=8.4 Hz, 2H), 3.61 (s, 3 H), 2.70 (t, J=7.6 Hz, 2H), 2.45 (t, J=7.7 Hz, 2H), 2.18 (s, 6H). ¹³C NMR (126 MHz, Chloroform-d) δ 1580, 142.4, 139.1, 135.4, 130.3, 126.8, 124.4, 109.7, 61.4, 45.6, 40.4, 33.9. HRMS (ESI-TOF) calculated for C₁₅H₂₀ClN₄O⁺ [M+H]⁺: 307.1320, observed 307.1314.

### Example 17

5-chloro-4-((4-(2-(dimethylamino)ethyl)phenyl)amino)-2-methylpyridazin-3(2H)-one (20). Following step 1 of the general procedure A, (4,5-dichloro-2-methylpyridazin-3(2H)-one (250 mg, 1.39 mmol, 1.0 eq.), 4-(2-dimethylamino -ethyl)aniline (214 mg, 1.26 mmol, 0.9 eq.), N,N-Diisopropylethylamine (487 μL, 2,79 mmol, 2.0 eq.)) and purification by flash column chromatography (CombiFlash Rf system: 4 g silica, DCM/methanol, 0-20% methanol, 20 minutes), product 20 (less polar fraction) was obtained as a yellow solid (37 mg, 10% yield). ¹H NMR (500 MHz, DMSO-d₆) δ 8.70 (s, 1H), 7.75 (s, 1H), 7.11 (d, J=8.4 Hz, 2H), 6.91 (d, J=8.3 Hz, 2H), 3.67 (s, 3H), 2.66 (t, J=7.7 Hz, 2H), 2.44 (t, J=7.8 Hz, 2H), 2.18 (s, 6H). ¹³C NMR (126 MHz, Chloroform-d) δ 158.0, 142.4, 139.1, 135.4, 130.3, 126.8, 124,4, 109.7, 61.4, 45.6, 40,4, 33.9, HRMS (ESI-TOF) calculated for C₁₆H₂₀ClN₄O⁺ [M+H]⁺307.1320, observed 307.1310.

### Example 18

5-((4-(2-aminoethyl)phenyl)amino)-4-bromo-2-methylpyridazin-3(2H)-one (21). Following the general procedure A, (4,5-dibromo-2-methylpyridazin-3(2H)-one (150 mg, 0.56 mmol, 1.0 eq.), tert-butyl (2-(4-amino-phenyl)-ethyl)carbamate (146 mg, 0.62 mmol, 1.1 eq.), N,N-Diisopropylethylamine (195 μL, 1.12 mmol, 2.0 eq.)), product 21 was obtained as a yellow solid (14 mg, 7% yield over two steps). ¹H NMR (500 MHz, DMSO-d₆) δ 7.48 (s, 1H), 7.24 (d, J=8.4 Hz, 2H), 7.18 (d, J=8.4 Hz, 2H), 3.61 (s, 3H), 2.82 (t, J=7.3 Hz, 2H), 2.68 (d, J=7.3 Hz, 2H). ¹³C NMR (126 MHz, DMSO-d₆) δ 157.3, 144.6, 136.8, 136.5, 129.6, 127.4, 124.1, 100.1, 42.7, 37.8 (one resonance obscured by solvent). HRMS (ESI-TOF) calculated for C₁₃H₁₆BrN₄O⁺ [M+H]⁺: 323.0502, observed 323.0488.

### Example 19

4-chloro-5-((4-(3-(dimethylamino)propyl)phenyl)amino)-2-methylpyridazin-3(2H)-one (22). Following step 1 of the general procedure A, 4,5-dichloro-2-methylpyridazin-3(2H)-one (552 mg, 3.08 mmol, 1.1 eq.), 4-(3-(dimethylamino)propyl)aniline (500 mg, 2.80 mmol, 1.0 eq.) and N,N-Diisopropylethylamine (975 μL, 5.60 mmol, 2.0 eq.) and purification by flash column chromatography (CombiFlash Rf system: 4 g silica, DCM/methanol, 0-20% methanol, 20 minutes). A portion of the product (91 mg, 10% crude yield) was further purified by reverse-phase HPLC (5-45% CH₃CN gradient over 30 minutes) to obtain product 22 as a white solid. ¹H NMR (500 MHz, DMSO-d₆) δ 8.69 (s, 1H), 7.75 (s, 1H), 7.10 (d, J=8.4 Hz, 2H), 6.92 (d, J=8.3 Hz, 2H), 3.67 (s, 3H), 2.57-2.52 (m, 2H), 2.23 (t, J=7.2 Hz, 2H), 2.14 (s, 6H), 1.68 (t, J=7.4 Hz, 2H). ¹³C NMR (126 MHz, DMSO-d6) δ 156.1, 138.2, 137.2, 136.6, 136.3, 127.6, 122.7, 111.0, 58.2, 44.9, 32.1, 28.6 (one resonance obscured by solvent). HRMS (ESI-TOF) calculated for C₁₃H₂₂ClN₄O⁺ [M+H]⁺: 321.1477, observed 321.1463.

### Example 20

5-((4-(aminomethyl)phenyl)amino)-4-chloro-2-ethylpyridazin-3(2H)-one (23). Following the general procedure A, (compound 26 (479 mg, 2.48 mmol, 1.0 eq.), tert-butyl (4-aminobenzyl)carbamate (607 mg, 2.73 mmol, 1.1 eq.), N,N-Diisopropylethylamine (864 μL, 4.96 mmol, 2.0 eq.)), product 23 was obtained as a yellow solid (62 mg, 9% yield over two steps). ¹H NMR (500 MHz, DMSO-d₆) δ 7.61 (s, 1H), 7.36 (d, J=8.4 Hz, 2H), 7.19 (d, J=8.4 Hz, 2H), 4.03 (q, J=7.2 Hz, 2H), 3.73 (s, 2H), 1.22 (t, J=7.1 Hz, 3H) (NH not observed). ¹³C NMR (126 MHz, DMSO-d₆) δ 156.5, 142.4, 140.9, 136.5, 128.0, 127.6, 123.8, 107.9, 46.2, 45.0, 13.5. HRMS (ESI-TOF) calculated for C₁₃H₁₆ClN₄O⁺ [M+H]⁺: 279.1007, observed 279.0995.

### Example 21

5-((4-(aminomethyl)phenyl)amino)-4-chloro-2-(prop-2-yn-1-yl)pyridazin-3(2H)-one (24). Following the general procedure A, (compound 27 (185 mg, 0.911 mmol, 1.0 eq.), tert-butyl (4-aminobenzyl)carbamate (223 mg, 1.00 mmol, 1.1 eq.), N,N-Diisopropylethylamine (317 μL, 1.82 mmol, 2.0 eq.)), a portion of the crude product was then purified by reverse-phase HPLC (5-45% CH₃CN gradient over 30 minutes) to obtain product 24 as a white solid (20 mg, 8% yield over two steps). ¹H NMR (500 MHz, DMSO-d₆) δ 8.95 (s, 1H), 8.20 (s, 3H), 7.66 (s, 1H), 7.48 (d, J=8.5 Hz, 2H), 7.31 (d, J=8.4 Hz, 2H), 4.94 (d, J=10.3 Hz, 1H), 4.04 (s, 2H), 2.18 (s, 2H). ¹³C NMR (126 MHz, DMSO-d₆) δ 156.8, 142.3, 138.6, 130.4, 130.1, 128.2, 125.5, 123,5, 108.7, 60.7, 41,8, 27.2. HRMS (ESI-TOF) calculated for C₁₄H₁₄ClN₄O⁺ [M+H]⁺: 289.0851, observed 298.0840.

### Example 22

5-((4(4-(aminomethyl)phenyl)amino)-4-chloro-2-isopropylpyridazin-3(2H)-one (25). Following the general procedure A, (compound 28 (270 mg, 1,31 mmol, 1.0 eq.), tert-butyl (4-aminobenzyl)carbamate (320 mg, 1.44 mmol, 1.1 eq.), N,N-Diisopropylethylamine (456 μL, 2.62 mmol, 2.0 eq.)), product 25 was obtained as a brown solid (65 mg, 17% yield over two steps). ¹H NMR (500 MHz, DMSO-d₆) δ 7.66 (s, 1H), 7.36 (d, J=8.4 Hz, 2H), 7.20 (d, J=8.4 Hz, 2H), 5.09 (hept, 6.6 Hz, 1H), 3.74 (s, 2H), 1.23 (d, J=6.7 Hz, 6H). ¹³C NMR (126 MHz, DMSO-d₆) δ 156.4, 141.9, 140.2, 136.6, 128.1, 127.4, 123.7, 107.8, 48.8, 44.8, 20.8 (NH₂ resonance not observed). HRMS (ESI-TOF) calculated for C₁₄H₁₈ClN₄O⁺ [M+H]⁺: 293.1164, observed 293.1153.

### Example 23

4,5-dichloro-2-ethylpyridazin-3(2H)-one (26). Following the general procedure B, (4,5-dichloropyridazin-3(2H)-one (1 g, 6.06 mmol, 1.0 eq.), ethyl bromide (680 μL, 9.09 mmol, 1.5 eq.), sodium hydride (160 mg, 6.67 mmol, 1.1 eq.)), product 26 was obtained as a white solid (525 mg, 45% yield). ¹H NMR (500 MHz, DMSO-d₆) δ 8.21 (s, 1H), 4.12 (d, J=7.3 Hz, 2H), 1.27 (t, J=7.3 Hz, 3H). ¹³C NMR (126 MHz, DMSO-d₆) δ 155.5, 136.6, 135.8, 132.8, 47.4, 13.1. HRMS (ESI-TOF) calculated for C₆H₇Cl₂N₂O⁺ [M+H]⁺: 192.9930, observed 192.9925.

### Example 24

4,5-dichloro-2-(prop-2-yn-1-yl)pyridazin-3(214)-one (27). Following the general procedure 8, (4,5-dichloropyridazin-3(2H)-one (2 g, 12.1 mmol, 1.0 eq.), propargyl bromide 80 wt. % in toluene (2.02 mL, 18.2 mmol, 1.5 eq.), sodium hydride (320 mg, 13.3 mmol, 1.1 eq.)), product 27 was obtained as a white solid (493 mg, 20% yield). ¹H NMR (500 MHz, DMSO-d₆) δ 8.25 (s, 1H), 4.90 (d, J=2.6 Hz, 2H), 3.42 (t, J=2.5 Hz, 1H), ¹³C NMR (126 MHz, DMSO-d₆) δ 155.1, 136.5, 136.4, 133.1, 77.5, 76.2, 41.9. HRMS (ESI-TOF) calculated for C₇H₅Cl₂N₂O⁺ [M+H]⁺: 202.9773, observed 202.9769.

### Example 25

4,5-dichloro-2-isopropylpyridazin-3(2H)-one (28). Following the general procedure B, (4,5-dichloropyridazin-3(2H)-one (1.5 g, 9.1 mmol, 1.0 eq.), isopropyl bromide (1.2 mL, 13.6 mmol, 1.5 eq.), sodium hydride (240 mg, 10.0 mmol, 1.1 eq.)), product 28 was obtained as a white solid (294 mg, 16% yield). ¹H NMR (500 MHz, DMSO-d₆) δ 8.25 (s, 1H), 5.15-5.00 (m, 1H), 1.29 (d, J=6.7 Hz, 6H). ¹³C NMR (126 MHz, DMSO-d₆) δ 155.4, 136.5, 135.6, 132.5, 50.6, 20.6.

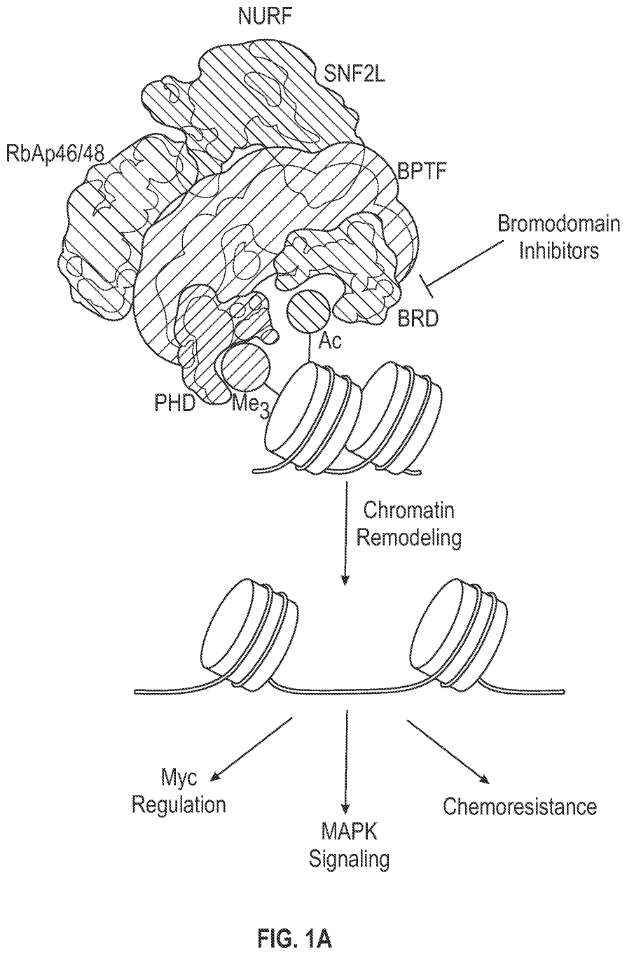

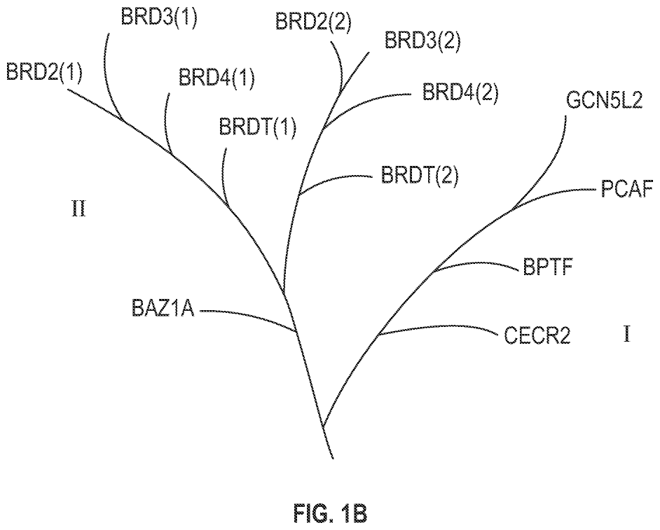

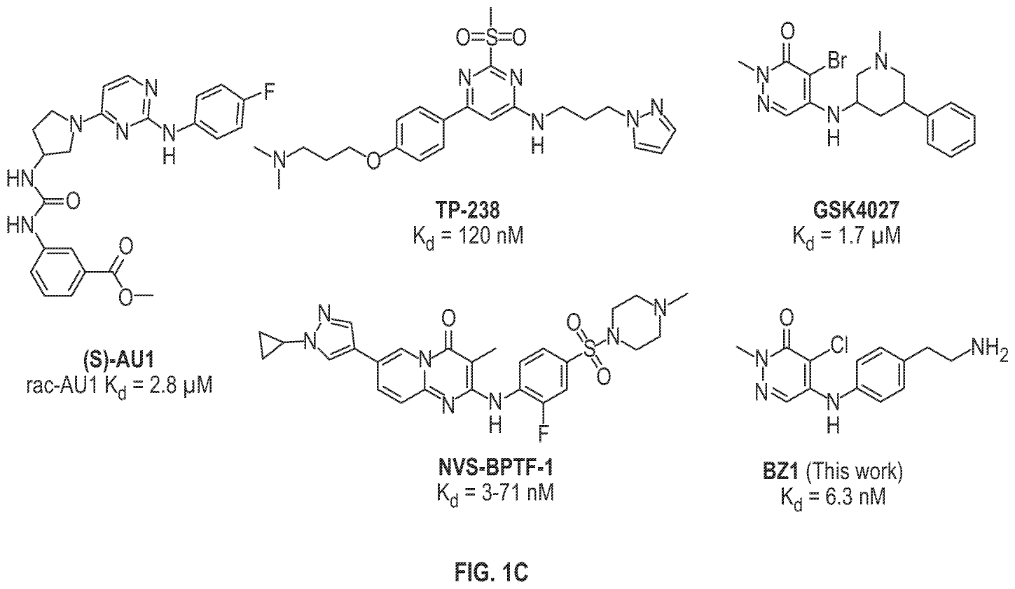

As a first step towards BPTF inhibitor development, several biophysical assays for BPTF ligand screening including a competitive inhibition AlphaScreen assay were recently cross-validated using an acetylated histone peptide and SPR binding experiments. Several compounds reported in the literature or online, including TP-238 and GSK4027, a PCAF/GCN5L2 inhibitor with off-target affinity for BPTF (Kd=1.7 μM, FIG. 1C) were used. Using these inhibitors and new fragment compounds, a number of small-molecule cocrystal structures with the BPTF bromodomain were reported. From these studies, GSK4027 was chosen for further analysis to establish design rules for inhibitor development.

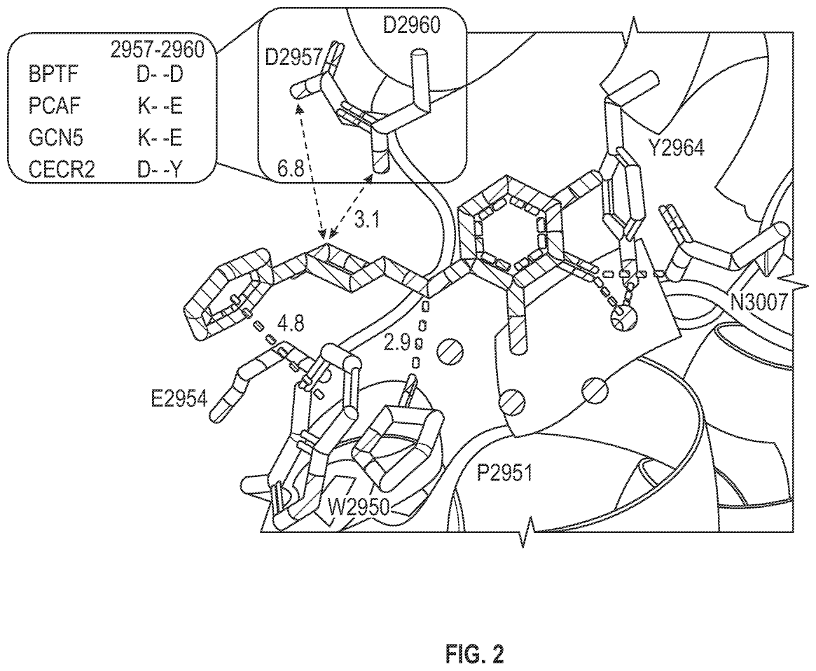

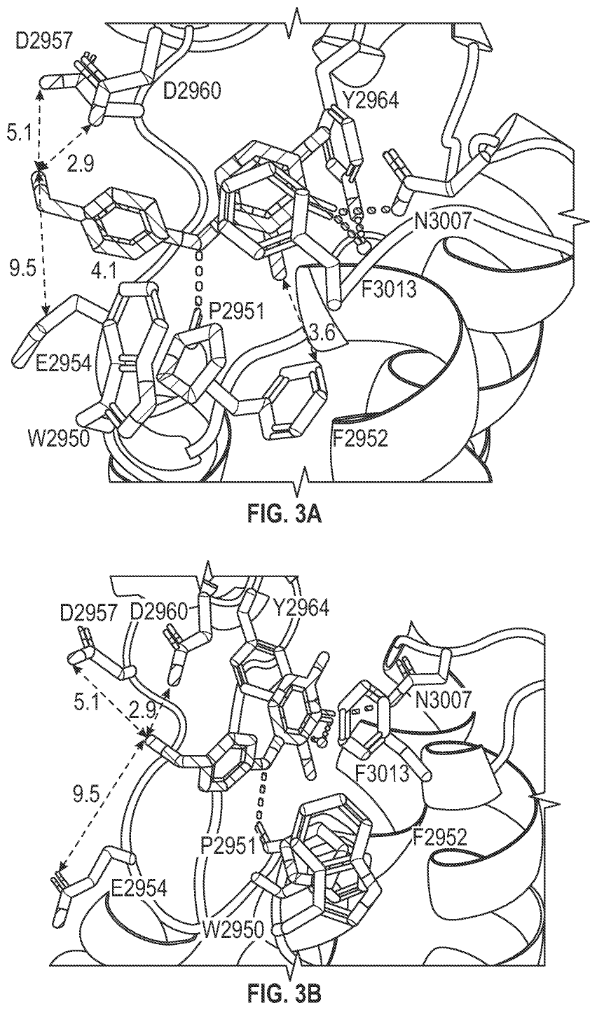

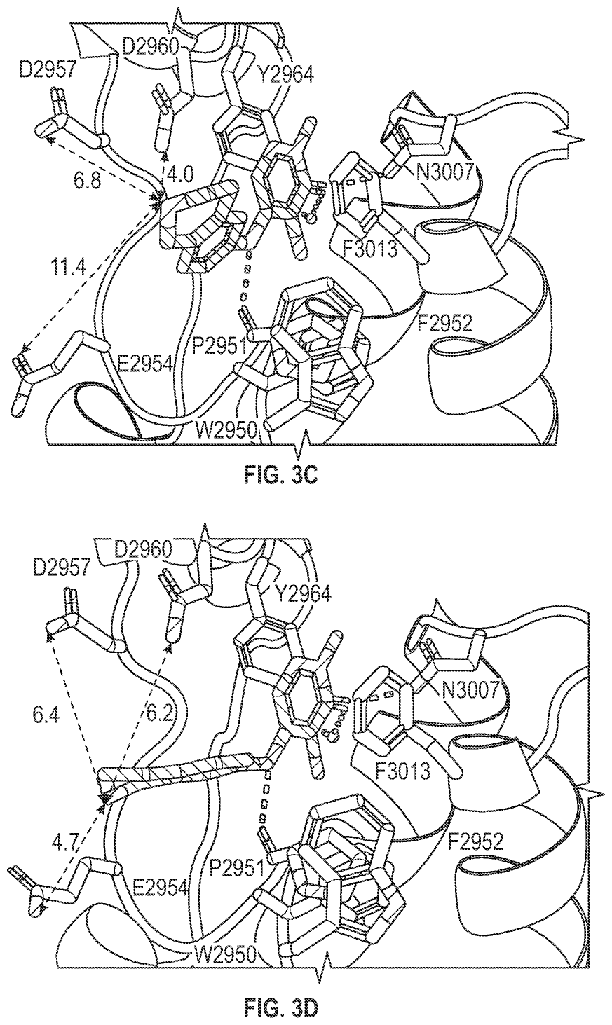

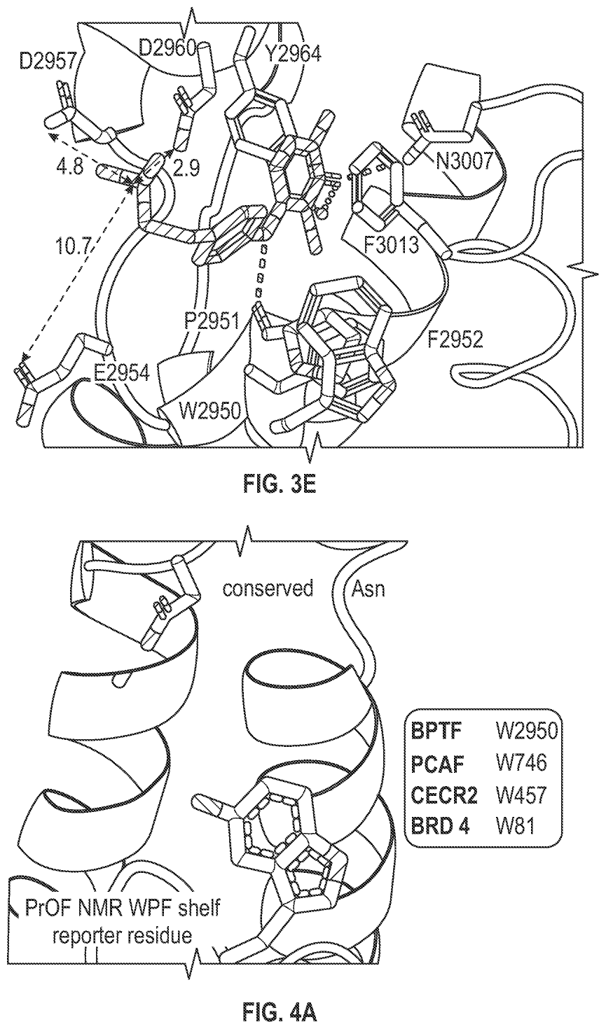

From a cocrystal structure of GSK4027 with BPTF bromodomain (FIG. 2), the carbonyl group acted as the acetyl lysine histone mimic, forming a hydrogen bond with N3007, and the bromine atom pointed into the binding pocket. The pyridazinone core formed Tr-stacking interactions with the gatekeeper residue F3013 (not shown here, see FIG. 3). The WPF shelf was engaged by hydrogen bonding to the P2951 backbone and W2950 by an edge-to-face interaction with the pendant phenyl ring, as previously shown with PCAF/GCN5L2.35 In addition, the acidic patch residues D2957 and D2960 were identified as key targets for inhibitor design (FIG. 2 inset). It was also shown that TP-238 could engage these side chains sup-porting this approach. Among class I bromodomains, BPTF is the only member with two acidic groups at this site so it was hypothesized that interactions with these side chains could improve both affinity and potentially selectivity for BPTF. It was anticipated that these interactions would provide multiple sites to fine-tune the potency and selectivity of the inhibitors described herein.

4,5-dichloropyridazinones were first tested, as a parent fragment of GSK4027 representing the acetylated lysine pharmacophore for the BPTF bromodomain. Protein-observed fluorine (PrOF) NMR was used as a sensitive biophysical assay to quantify weak interactions with BPTF, using a fluorine-labeled tryptophan at W2950.37 In this experiment, the protein resonance showed a significant dose-dependent shift and broadening below 100 μM of the compound. A dose-dependent chemical shift perturbation at low concentrations was consistent with significant affinity of this pharmacophore for BPTF. Encouraged by the apparent potency of the starting fragment and the relatively facile synthesis towards elaborated compounds (Scheme 1), a library of pyridazinone-containing aliphatic amines similar to GSK4027 (Table 1) was generated and tested with BPTF using PrOF NMR and a competitive inhibition AlphaScreen assay.

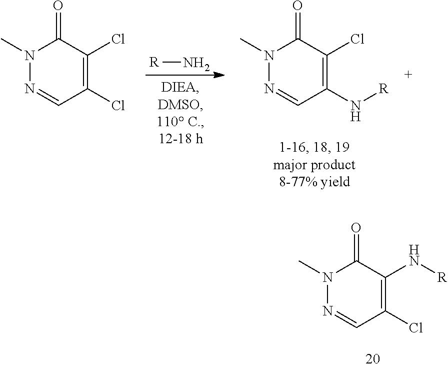

Aliphatic pyridazinone series. The initial synthesis started with a nucleophilic aromatic substitution reaction with various aliphatic amines, generating the desired compounds 1-5 as the major isomers (Table 1). Initial characterization of the affinity of 1-3 was recently reported. The IC₅₀ values ranged from 7.7-31 μM. To gain structural insight, cocrystal structures of compounds 1-4 were acquired with BPTF. These structures supported the importance of the exocyclic amine in maintaining the hydrogen bonding interaction with the backbone carbonyl of P2951, similar to GSK4027. However, the ring size and position of the endocyclic amine group did not significantly impact the affinity of the compounds and accessibility to the acidic D2957 and D2960 side chains. Interestingly, these crystal structure revealed a water-mediated hydrogen bond with E2954, an interaction not previously explored in BPTF inhibitor design.

TABLE 1
SAR with aliphatic pyridazinones and BPTF

| | | BPTF | |
|---|---|---|---|
| | | AlphaScreen | |
| | R | IC₅₀ (μM) | L.E. |
| GSK | | 1.5 ± 0.2³⁶ | 0.35 |
| 4027 | | | |
| 1 | 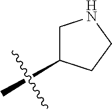 | 10 ± 2³⁶ | 0.45 |
| 2 | 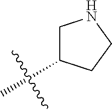 | 31³⁶ | 0.41 |
| 3 | 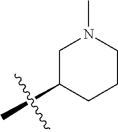 | 19³⁶ | 0.38 |
| 4 | 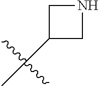 [SMILES: *C1CNC1] | 8.7 | 0.49 |
| 5 | 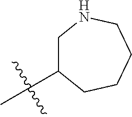 [SMILES: *C1CCCCNC1] | 7.7 | 0.41 |

AlphaScreen values were an average of two technical replicates with N = 1, except for GSK4027 and 1 which were averages of six and three experimental replicates, respectively.

Aromatic amine substituted pyridazinones. Based on the hypothesis that the N—H interaction with P2951 was important for the affinity of pyridazinone inhibitors described herein, it was proposed that the more acidic aniline N—H could be a stronger H-bond donor compared to aliphatic amines. Therefore, in a second series of inhibitors, aromatic amine-substituted pyridazinones (Table 2) were investigated. The aniline-substituted compound 6 (previously reported as a PCAF and BRD9 inhibitor with affinities of 10 μM and 2.5 μM respectively) demonstrated a 10-fold improvement in affinity and higher ligand efficiency (L.E.) compared to previous aliphatic amine analogues.

The effects of electron-donating and -withdrawing substituents on the aromatic ring were compared and it was found that the para-fluoro group (8) led to an improved affinity compared to a para-amino group (7). This observation was consistent with the importance of the hydrogen-bonding interaction with the P2951 backbone, which would be assisted by an electron-withdrawing group on the ring and the more acidic character of the conjugate acid of the anilinic NH. In agreement with this data, compound 9, containing a benzylic amine group attached to the pyridazinone core, was also a weaker binder of BPTF compared to 6 and 7. Interestingly, an analogue of 9 was also recently identified as the starting fragment for BRD9 inhibitors, with pIC₅₀=5.7 and 6-fold selectivity over PCAF.

TABLE 2
Aniline-substituted pyridazinones and substituent effects for
binding to BPTF

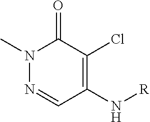

| | | BPTF | |
|---|---|---|---|
| | | AlphaScreen | |
| | R | IC₅₀ (μM) | L.E. |
| 6 | 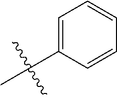 [SMILES: *c1ccccc1] | 0.95 | 0.51 |
| 7 | 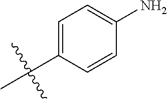 [SMILES: *c1ccc(N)cc1] | 3.2 | 0.44 |
| 8 | 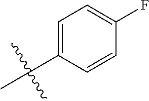 [SMILES: *c1ccc(F)cc1] | 0.70 | 0.49 |
| 9 | 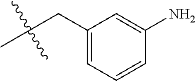 [SMILES: *Cc1cccc(N)c1] | 11 | 0.40 |

AlphaScreen values were an average of two technical replicates, N = 1.

Aromatic pyridazinones: Effect of basic group substitution. In a further round of SAR, based on an acidic patch hypothesis, different amine substitutions on the aromatic ring were investigated for engaging D2957 and D2960 (Table 3). Encouragingly, extending the NH₂ group by just one methylene (from compound 7 to 10) resulted in a ˜10-fold improvement in potency, with IC₅₀ values of 0.29 μM and 0.31 μM, and ligand efficiencies of 0.50 and 0.44 respectively for compounds 10 and 11. This gain in affinity was attributed to a potential electrostatic interaction between the amine group and the aspartate side chains of BPTF. Such an interaction was also consistent with the loss in affinity observed when the amine was removed (14) or the positive charge neutralized via acetylation (17).

The effect of the position of the amine group was also explored, expecting to see significant differences based on which orientation of the group was more favorable for engaging D2957 and D2960. Surprisingly, the regioisomers 12 and 13 displayed similar affinities, which were comparable to 10. In this series, 15, where the amine was no longer restricted in a ring, was a weaker binder compared to 13. While compound 16 showed a high affinity, it was obtained in the lowest synthetic yield and was also previously reported to have affinity for an off-target bromodomain, BRD9.

TABLE 3
SAR with aromatic pyridazinones containing different basic groups
substitutions for binding to BPTF

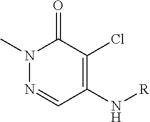

| | | BPTF | |
|---|---|---|---|
| | | AlphaScreen | |
| | R | IC₅₀ (μM) | L.E. |
| 10 | 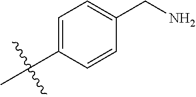 [SMILES: *c1ccc(CN)cc1] | 0.29 ± 0.08 | 0.50 |
| 11 | 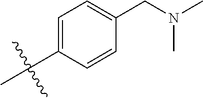 [SMILES: *c1ccc(CN(C)C)cc1] | 0.31 | 0.44 |
| 12 | 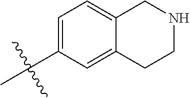 [SMILES: *c1ccc2c(c1)CCNC2] | 0.25 | 0.45 |
| 13 | 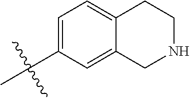 [SMILES: *c1ccc2c(c1)CNCC2] | 0.37 | 0.44 |
| 14 | 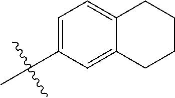 [SMILES: *c1ccc2c(c1)CCCC2] | 3.9 | 0.37 |
| 15 | 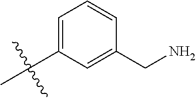 [SMILES: *c1cccc(CN)c1] | 0.80 | 0.46 |
| 16 | 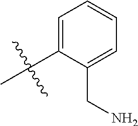 [SMILES: *c1ccccc1CN] | 0.22 | 0.50 |
| 17 | 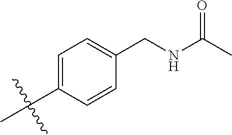 [SMILES: *c1ccc(CNC(C)=O)cc1] | 0.97 | 0.39 |

X-ray crystallography was used to obtain structural information that could account for the similar affinities of amine analogues 10-13 (FIGS. 3A-3E). Similar to the aliphatic amines, all cocrystal structures displayed the canonical hydrogen bonding with N3007 and water-mediated hydrogen bonding with Y2964 and a key hydrogen bond with P2951. The phenyl Groups were 3.8-5.0Å from W2950, which could contribute to the higher potency of an aromatic series over the aliphatics, forming a CH-π interaction. The amine group on compound 10 was 2.9Å away from D2960, which could explain the improved affinity over compounds 7, 14, and 17. While compound 12 retained these interactions, it was surprising that the different orientation of the basic group in compound 13 led to an interaction with E2954. Therefore, the improved affinities of aromatic pyridazinone series compared to the aliphatics can be attributed to an additional aromatic interaction with the WPF shelf, strengthened H-bonding interactions with P2951, and differential engagement of side chains in a potential acidic triad (D2960, D2957 and E2954), depending upon the relative orientations of the amine moieties.

Based on this structural analysis, it was proposed that extending the amine group could lead to a further improvement in potency as D2957 was 5.1-6.8Å away from the basic Group on the compounds described herein. In support of this, compound 18, with just an additional methylene, showed a 4-fold improvement in affinity over 10, with an AlphaScreen IC₅₀ of 67 nM and ligand efficiency 0.51 (Table 4). The N,N-dimethyl analogue, 19, was slightly less potent, but may improve cellular permeability due to fewer hydrogen bond donors.⁴¹ To test the importance of the H-bond with P2951, the 4-position regioisomer 20 was also isolated. Supporting this interaction is a significantly reduced potency (IC₅₀=10 μM). For future cellular studies, 20 can serve as an important negative control compound (140-fold weaker affinity than 18).

To validate the designs, a cocrystal structure of BPTF with 19, a close analogue of inhibitor 18 (FIG. 3E) was obtained. In this case, the amino group was now within 5Å of D2957 and D2960, supporting the enhanced affinity for engaging either acidic group via electrostatic interactions. An overlay of the apo structure with 19 indicated very little movement of acidic residues.

TABLE 4
Aromatic pyridazinones with extended basic group

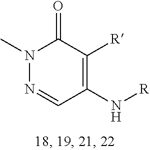

| | | | BPTF | |
|---|---|---|---|---|
| | | | Alpha | |
| | | | Screen | |
| | R | R′ | IC₅₀ (μM) | L.E. |
| 18 (BZ1) | 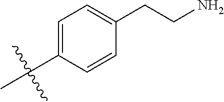 [SMILES: *c1ccc(CCN)cc1] | Cl | 0.067 ± 0.01 | 0.51 |
| 19 | 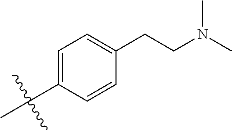 [SMILES: *c1ccc(CCN(C)C)cc1] | Cl | 0.17 | 0.44 |
| 20 | 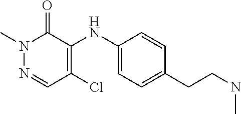 | Cl | 10 | 0.32 |
| 21 | 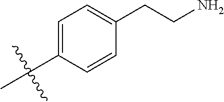 [SMILES: *c1ccc(CCN)cc1] | Br | 0.036 ± 0.008 | 0.53 |
| 22 | 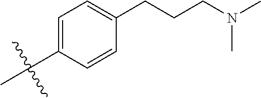 [SMILES: *c1ccc(CCCN(C)C)cc1] | Cl | 0.056 ± 0.01 | 0.45 |

AlphaScreen values were an average of two technical replicates, with N = 1 except for 18 (BZ1) which was an average of seven experimental replicates and 21-22 which were averages of three experimental replicates.

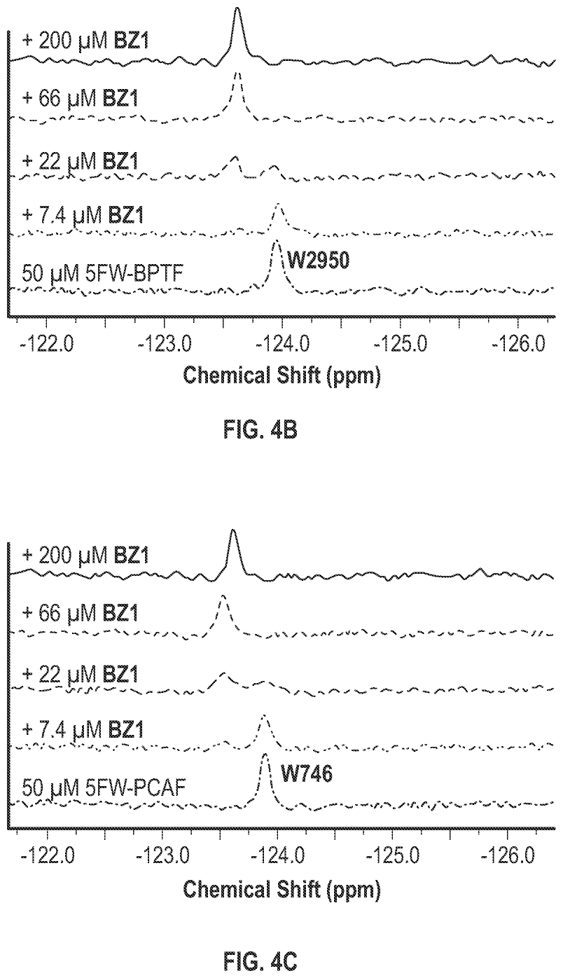

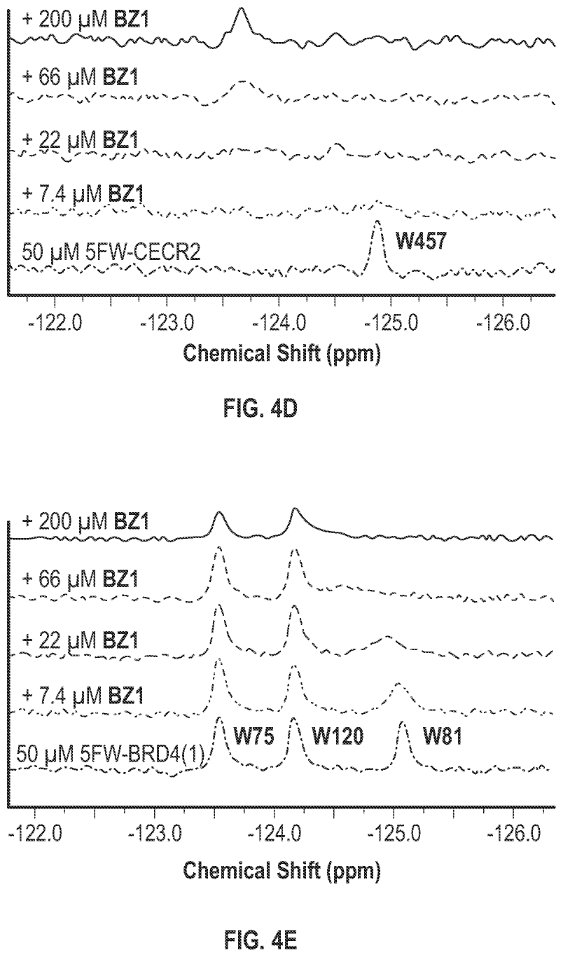

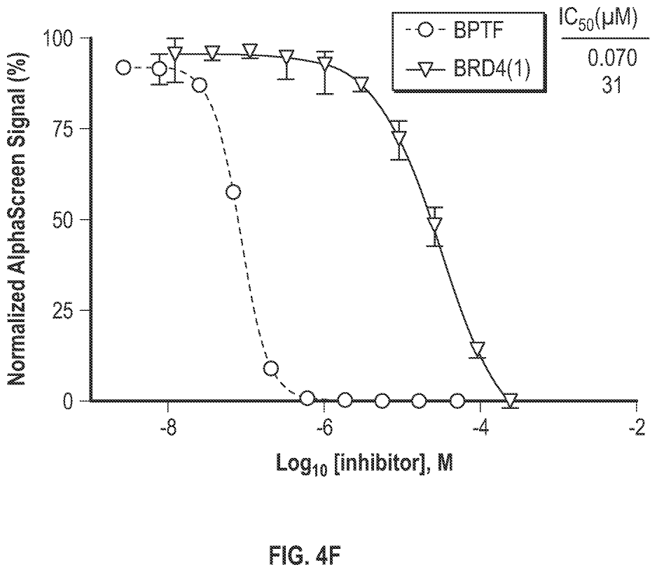

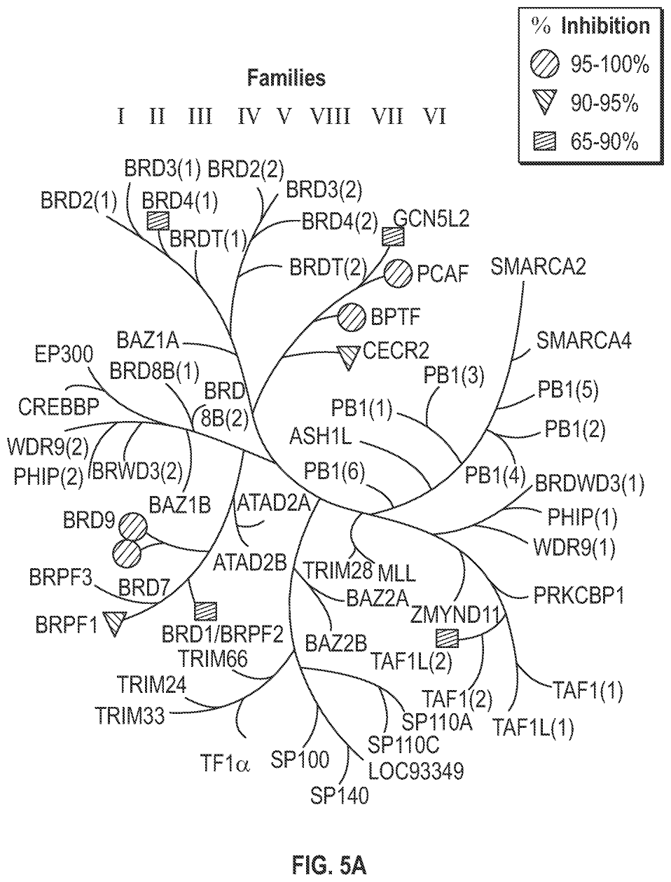

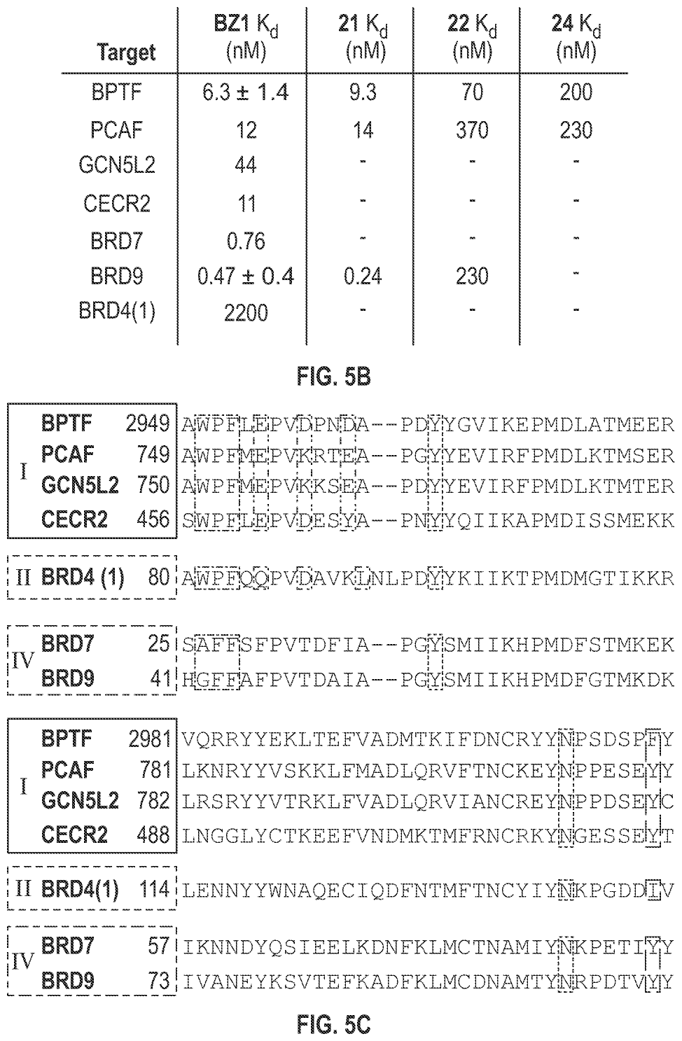

Selectivity Profile of compound 18 (BZ1) with bromodomain families. A preliminary assessment of the selectivity of compound 18, referred to as BZ1 here onwards, was conducted using PrOF NMR assay (FIGS. 4B-4D). The tryptophan residue in the WPF shelf of three class I bromodomains, BPTF, PCAF, CECR2 and one class II bromodomain, BRD4(1) were fluorine-labelled (FIG. 4A) and the chemical shift perturbation on titrating in BZ1 was observed. For both BPTF and PCAF, a slow exchange regime stoichiometric titration was observed, with the bound and unbound resonances resolved at sub-stoichiometric concentrations of BZ1. CECR2 showed intermediate chemical exchange, indicating that BZ1 was a weaker binder for CECR2 compared to BPTF and PCAF in this assay. Importantly, BRD4(1) demonstrated fast-intermediate exchange, showing qualitatively that BZ1 was the weakest inhibitor for BET bromodomains under study here. AlphaScreen assay was also used to quantify the affinity for BRD4(1) as a representative member of the BET family (FIG. 4E). In this experiment, BZ1 was found to be 400-fold selective for BPTF over BRD4(1), consistent with the PrOF NMR results. Selectivity over the BET family is important for non-BET chemical probes because BET inhibition shows a strong cellular phenotype which can mask any BPTF-dependent effects. in both BPTF and PCAF, an acidic residue is present in the acidic dyad, whereas in CECR2 and BRD4(1) the 3D equivalent is a tyrosine or leucine, respectively, and may account for some of the apparent selectivity differences (FIGS. 2 and 5C). Moreover, PrOF NMR data also demonstrated that BZ1 with a clogP=1.6 can be titrated at high micromolar concentrations at 1% DMSO and shows dose dependence, indicating good solubility. The solubilities of BZ1, 19, and 20 were further confirmed up to 100 μM at 0.1% DMSO using UV-Vis spectroscopy.

Based on the preliminary assessment of BPTF selectivity and affinity of BZ1 by PrOF NMR and AlphaScreen competition assays, the ligand was characterized using a commercial BROMOscan assay. Using this assay, the K_{d} of BZ1 for BPTF was determined to be 6.3 nM (FIG. 5B). Given the low concentration of ligand and protein used, AlphaScreen can be used to estimate K values as was previously the case for characterizing BRD4-ligand interactions, however the assay for BPTF may slightly underestimate the affinity. Given the high affinity of BZ1, its selectivity was measured against a panel of 32 representative bromodomains with a one-point measurement in the same assay format. These assays were performed at 140 nM, approximately twenty times above the K_{d} of BZ1 for BPTF (FIG. 5A). Consistent with PrOF NMR and AlphaScreen results, the BET family proteins, were weakly inhibited with the highest estimated affinity for BRD4(1) (71% inhibition). For Class I, bromodomains, BPTF and PCAF were significantly inhibited as expected (100% inhibition), with lower levels of inhibition for CECR2 and GCN5L2. Although, BRD7 and BRD9 lack acidic residues corresponding to the acidic triad, they were also strongly inhibited (99%). Recently reported pyridazinone-based inhibitors also bind to these proteins and BRD9 was reported as the closest off-target for TP-238. These studies supported good on target-BPTF inhibition, and identified several off-targets bromodomains for a more quantitative selectivity analysis.

Given that these measurements were only estimates of affinity, a full titration was carried out for five additional bromodomains (FIG. 5B). In this case, a 350-fold selectivity was obtained over BRD4(1). However, the selectivity over class I bromodomains, PCAF, CECR2, and GCN5L2 was reduced. The affinity for BRD7 and 9 was stronger than expected (K_{d}=0.76 and 0.47 nM respectively) and represents an important off-targets for future inhibitor designs. During the course of preparing this manuscript, a new BPTF inhibitor was reported with the highest affinity of 428 nM. However selectivity studies against BRD9 and class I bromodomains were not conducted in this study to allow comparisons. Currently the ability to potently inhibit both the SWI/SNF and NURF nucleosome remodeling complexes have yet to be explored and may provide a novel mechanism for therapeutic applications.

As an initial evaluation of two additional analogs to improve activity, 21 and 22 were synthesized and tested. 21 is an analogue of BZ1 which replaces the chloro group with a bromine atom, analogous to GSK4027. 22 is an analog of 19 which extends the amino group by one additional methylene to further engage D2957. In the case of 21, there was a small but significant improvement in affinity by AlphaScreen relative to BZ1 and a 3-fold increase in potency of 22 relative to 19. Theft affinity and selectivity by BROMOscan were also measured. While the K_{d} of 22 was weaker (K_{d}=70 nM), both BRD9 affinity and PCAF affinity were weakened more significantly and now result in a modest selectivity over BRD9 and further selectivity over PCAF. These results support the design strategy for targeting the two acidic residues of BPTF to enhance the selectivity of the inhibitor series.

Exploring the SAR at the pyridazinone N—CH₃. As a second attempt to improve selectivity and/or affinity, in the final SAR series, the N—CH₃ position on the pyridazinone core was investigated. Using the cocrystal structure reported for NVS-BPTF-1, it was hypothesized that the cyclopropyl-substituted pyrazole ring may contribute to the affinity and selectivity for BPTF. In the scaffold, the analogous position would be the R′ substituent in Table 5. It was observed that small alkyl groups and a propargyl group were tolerated at that position, albeit with no improvement in affinity. However, all the analogues retained their selectivity over BRD4(1). The affinity of 24 was further characterized with BPTF and PCAF using BROMOscan, obtaining Kd values of 200 nM and 230 nM respectively. The alkyne group can serve as a useful click-chemistry handle for further modifications of the pyridazinone scaffold.

TABLE 5
Aromatic pyridazinones with 2-position N-alkyl substituents

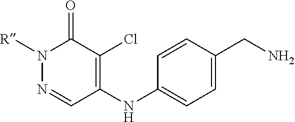

| | | BPTF | BRD4(1) |
|---|---|---|---|
| | | AlphaScreen | AlphaScreen |
| | R″ | IC₅₀ (μM) | IC₅₀ (μM) |
| 10 | CH₃ | 0.29 | 42 |
| 23 | 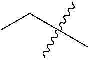 [SMILES: *CC] | 0.55 | 35 |
| 24 | 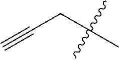 [SMILES: *CC#C] | 0.38 | 71 |
| 25 | 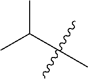 [SMILES: *C(C)C] | 0.79 | NB |

AlphaScreen values were an average of two technical replicates with N = 1.
NB indicates that the compound was non-binding up to 250 μM.

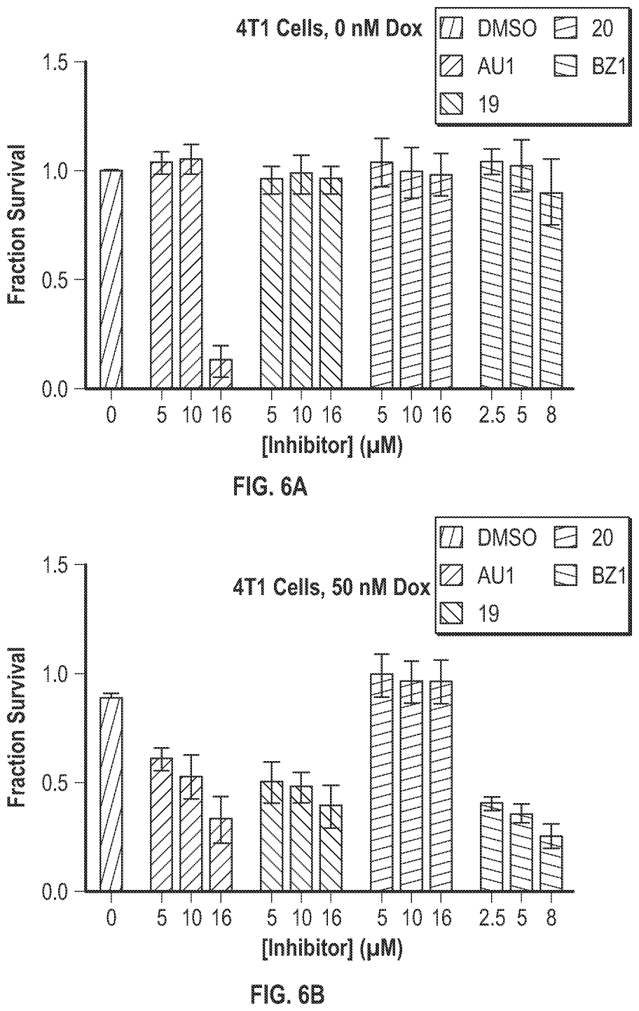

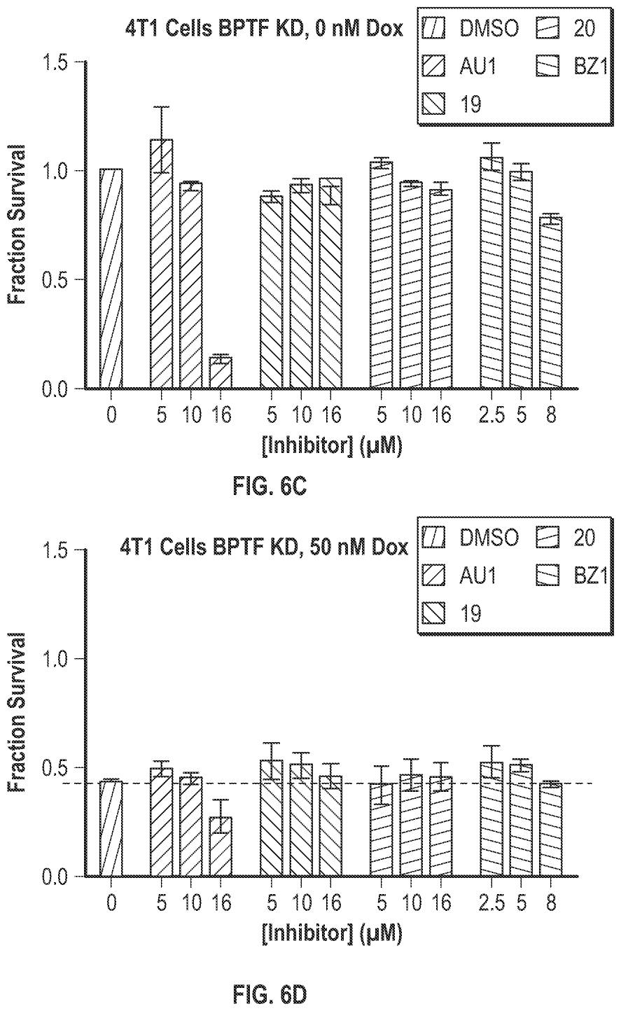

Enhancing toxicity of chemotherapeutics in a model breast cancer cell line. With potent inhibitors in hand, an initial assessment of cellular activity prior to further selectivity optimization was conducted. BPTF has been implicated in resistance to chemotherapeutics for treating hepatocellular carcinoma, and BRAF inhibitors for melanoma therapy. BPTF suppression of Topoisomerase 2 poisons has been identified previously, including doxorubicin and etoposide, whose cytotoxic activity was enhanced with BPTF knockdown or bromodomain inhibition with AU1. While knockdown of BPTF in 4T1 mouse breast cancer cells does not exhibit toxicity on its own, AU1 treatment exhibited toxicity at higher concentrations consistent with an off-target effect. Several pyridazinones were tested and were found to be well-tolerated by the 4T1 cells up to mid-micromolar concentrations, with the exception of BZ1 which started to exhibit some toxicity at 8 μM (% survival=56 and 89% in two separate experiments; FIG. 6A). BZ1, 19 and a regioisomer control, 20, were further used for combination treatment with doxorubicin at concentrations lacking significant toxicity with inhibitor alone. (FIGS. 6A-6B) BZ1 and 19 sensitized 4T1 cells to doxorubicin, exhibiting sensitization similar to BPTF sh RNA knockdown levels, while 20 did not. A separate dose dependence experiment showed BZ1 maintained strong biological effects down to 2.5 μM while 19 was 2-4-fold less effective. This result is consistent with the weaker affinity of 19 towards the BPTF bromodomain. It remains unclear if the lack of an effect at concentrations closer to the inhibitors' biochemical potencies are due to a lack of cellular uptake, or if alternate mechanism are also important such as engagement of additional BPTF domains with chromatin. As a control for off-target effects no further toxicity was observed when BPTF knockdown cells were treated with BPTF pyridazinone inhibitors and doxorubicin at these concentrations despite the high BRD9 affinity (FIGS. 6C-6D). Additional toxicity was observed for AU1 at the highest concentrations tested. Together, these results are consistent with an on target BPTF bromodomain inhibition effect of a new inhibitor class.

Pyridazinones effect on BPTF target genes. As a final evaluation of BPTF-dependent cellular effects, the effects of 19 on several potential BPTF target genes was tested. 19 was chosen due to its low level of toxicity in 4T1 cells, and its regioisomer control 20. AU1 was also tested as a second control for BPTF inhibition. Given the lack of BPTF inhibitors, few-BPTF dependent genes have been validated for bromodomain inhibition and prior work has shown BPTF bromodomain inhibitors do not replicate all genes affected by BPTF depletion.

It's been previously shown that BPTF inhibition was associated with alteration to lineage commitment and stem cell maintenance. Loss of BPTF expression in a mixed population of Krt5-expressing mammary stem cells induced differentiation, a process that was accompanied by changes to chromatin accessibility and altered gene expression activation. The effects of the BPTF inhibitors described herein were investigated in mammary luminal cells. The murine Eph4 cell line was used, an immortalized, normal-like system previously shown to activate molecular process of luminal cell differentiation, and were responsive to AU1 treatment. Here, Eph4 cells were treated with AU1 (5 μM), 19 (5 μM), and its regioisomer control 20 (5 μM), followed by either apoptosis analysis or RNA extraction. The mRNA levels of the three genes were analyzed via RT-qPCR based on prior analysis of BPTF knockout studies in mammary epithelial luminal cells which included two highly upregulated genes, Stratifin (Sfn), and SmaII proline rich protein 1A (Sprr1a). Also analyzed were Myc levels given prior reports on BPTF regulation, although prior knockout data did not show a statistically significant effect.

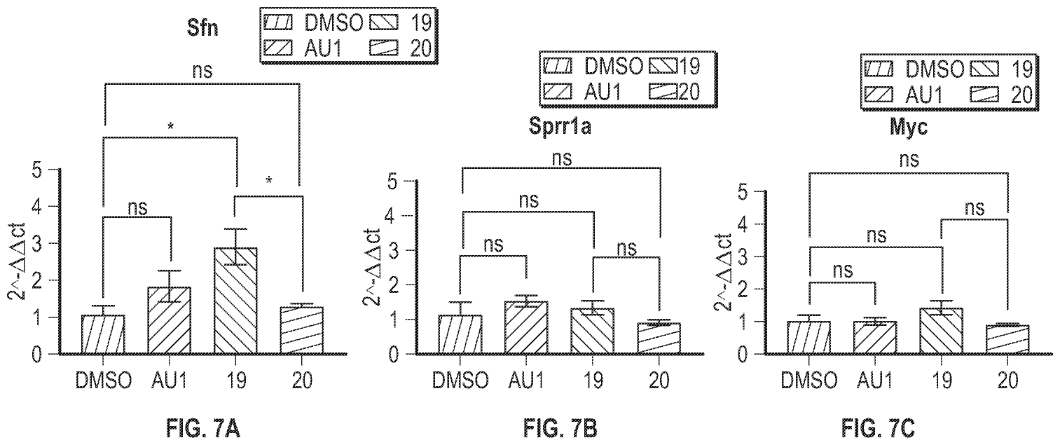

Compound 19 treatment led to minimal toxicity against Eph4 cells and induced statistically significant increase in Sfn which was not significantly affected by 20 (FIG. 7A). AU1 upregulated Sfn but did not reach a high enough level of statistical significance. Conversely, Sprr1a was not significantly affected by any treatment (FIG. 7B). This result suggests potential differential effects between bromodomain inhibition and whole protein knockout. Myc levels were also unaffected relative to DMSO treatment (FIG. 7C). Unaffected Myc levels are consistent with a lack of caspase activation by 19. This preliminary analysis shows that compound 19 treatment can induce cellular effects in at least one gene associated with BPTF knockout studies, and warrants further investigation. A limitation of the analysis is the comparison to BPTF knockout cells from a mixed population, and a need for analysis with more selective bromodomain inhibitors which is the focus of future work.

These results describe the development of new BPTF inhibitors based on a pyridazinone scaffold with the lead molecule BZ1 having a high affinity for BPTF (K_{d}=6.3 nM) and >350-fold selectivity over the BET family, making it the most potent inhibitor for the BPTF bromodomain in the published literature. Cocrystal structures of analogues establish a framework of structure-based design that can aid future efforts in rational development of chemical probes and to engineer selectivity over off-target bromodomains as follows:

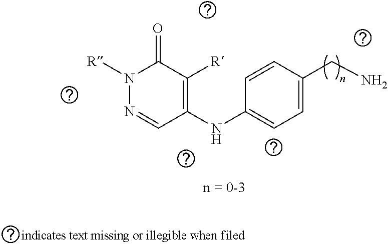

Molecule 22 is one such example for reducing affinity towards BRD7/9. As not all bromodomain inhibitors exhibit cellular effects, breast cancer cell lines were used herein to show that the inhibitors described herein have on-target activity for BPTF and sensitize to the chemotherapy drug doxorubicin. Their activity is significantly improved relative to AU1, which is less effective with a sharp toxicity profile starling above 16 μM. The high potency, solubility and ligand efficiency (0.51) of BZ1 makes it a suitable lead for further medicinal chemistry optimization and the development of new chemical biology tools.

1. A compound of the formula (I): 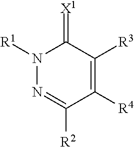
or a pharmaceutically acceptable salt thereof;
wherein:
X¹ is O, NR⁵ or S, wherein R⁵ is H, alkyl, arylalkyl or OR⁶, wherein R⁶ is H, alkyl, or arylalkyl;
R¹ and R² are each independently H, alkyl, alkynyl, cycloalkyl or heterocyclyl;
R³ is halo; and
R⁴ is —NHR⁷, wherein R⁷ is aryl, arylalkyl, heterocyclyl or heterocyclylalkyl; or
R⁴ is halo; and
R³ is —NHR⁷, wherein R⁷ is aryl, arylalkyl, heterocyclyl or heterocyclylalkyl;
wherein when R³ is chloro, R⁷ is not pyrrolidinyl or piperidinyl.

2. The compound of claim 1, or a pharmaceutically acceptable salt thereof, wherein R³ is halo and R⁴ is —NHR⁷ or R³ is —NHR⁷ and R⁴ is halo.

3. (canceled)

4. (canceled)

5. The compound of claim 1, or a pharmaceutically acceptable salt thereof, wherein R⁷ is heterocyclyl.

6. The compound of claim 1, or a pharmaceutically acceptable salt thereof, wherein R⁷ is a four-, five- or six-membered heterocyclyl group.

7. (canceled)

8. The compound of claim 1, or a pharmaceutically acceptable salt thereof, wherein the compound of the formula (I) is a compound of the formula (Ia) or (Ib): 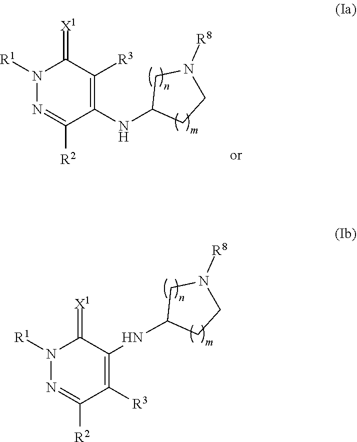
or a pharmaceutically acceptable salt thereof;
wherein R⁸ is H, alkyl or arylalkyl;
m is 0, 1, 2 or 3; and
m is 0, 1, 2 or 3.

9. (canceled)

10. (canceled)

11. (canceled)

12. The compound of claim 8, or a pharmaceutically acceptable salt thereof, wherein the compound of formula (Ia) or (Ib) is a compound of the formula: 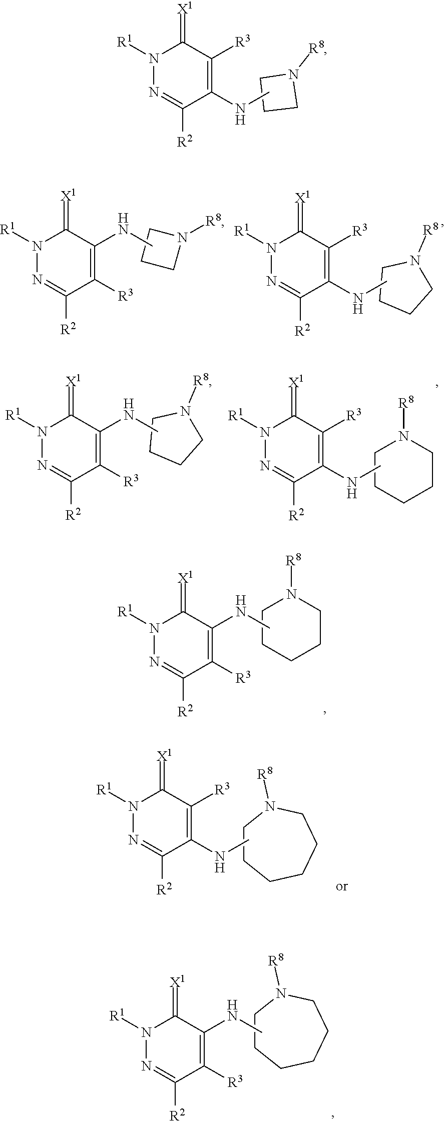
or a pharmaceutically acceptable salt thereof.

13. (canceled)

14. (canceled)

15. The compound of claim 1, or a pharmaceutically acceptable salt thereof, wherein R⁷ is aryl or arylalkyl.

16. (canceled)

17. The compound of claim 15, or a pharmaceutically acceptable salt thereof, wherein the compound is a compound of the formula (Ic) or (Id): 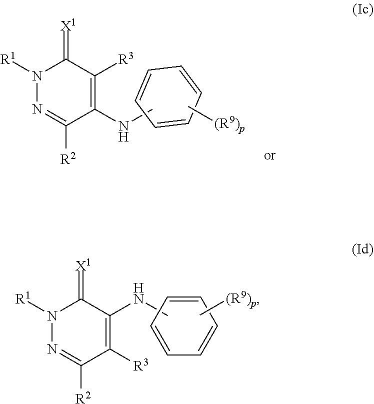
or a pharmaceutically acceptable salt thereof;
wherein:
p is 1, 2 or 3; and
each R⁹ is independently H, alkyl, alkoxy, amino, aminoalkyl, amido, amidoalkyl or two R⁹ groups located on adjacent carbon atoms can, together with the atoms to which they are attached, form a heterocyclyl or a cycloalkenyl group.

18. The compound of claim 15, or a pharmaceutically acceptable salt thereof, wherein the compound is a compound of the formula: 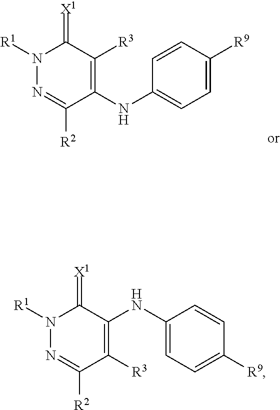
or a pharmaceutically acceptable salt thereof;
wherein:
R⁹ is independently H, alkyl, alkoxy, amino, aminoalkyl, amido or amidoalkyl.

19. The compound of claim 15, or a pharmaceutically acceptable salt thereof, wherein R⁷ is a group of the formula: 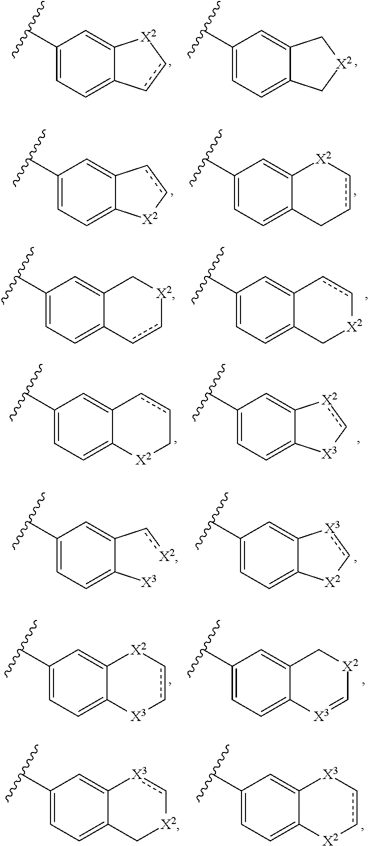
wherein the dashed line can represent a double bond; X² is CH₂, O or NR¹⁰, wherein R¹⁰ is absent when a double bond is present; and X³ is CH₂, O or NR¹⁰.

20. The compound of claim 19, or a pharmaceutically acceptable salt thereof, wherein R⁷ is:

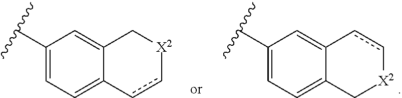

21. The compound of claim 19, or a pharmaceutically acceptable salt thereof, wherein R⁷ is a group of the formula:

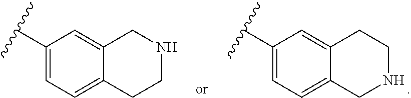 [SMILES: *c1ccc2c(c1)CNCC2 ; *c1ccc2c(c1)CCNC2]

22. The compound of claim 1, or a pharmaceutically acceptable salt thereof, wherein X¹ is O.

23. The compound of claim 1, or a pharmaceutically acceptable salt thereof, wherein R¹ is alkyl or alkynyl.

24. The compound of claim 1, or a pharmaceutically acceptable salt thereof, wherein R² is H.

25. The compound of claim 1, or a pharmaceutically acceptable salt thereof;
wherein the compound has the formula:

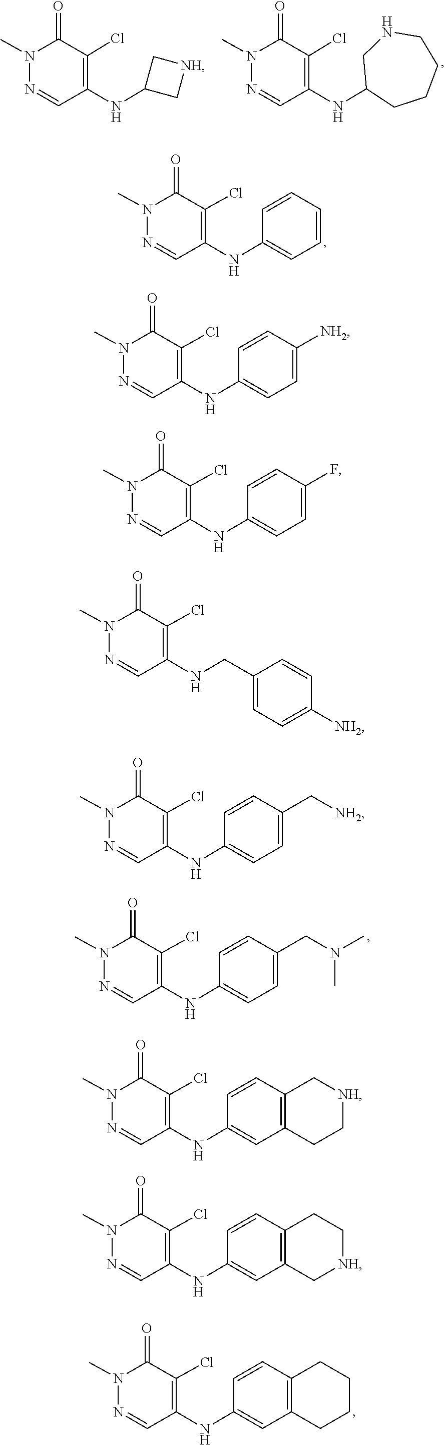

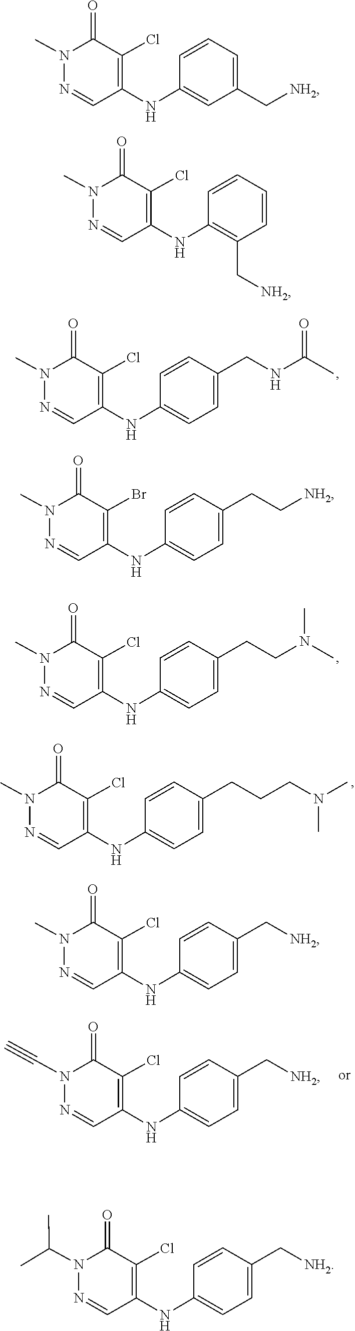

26. A pharmaceutical composition comprising one or more compounds of claim 1, or a pharmaceutically acceptable salt thereof, and one or more pharmaceutically acceptable excipients.

27. A method for treating cancer, the method comprising administering a therapeutically effective amount of at least one compound of claim 1, or a pharmaceutically acceptable salt thereof to a subject in need thereof.

28. The method of claim 27, wherein the cancer is breast cancer, non-small-cell lung cancer, colorectal cancer or high-grade gliomas.

29. The method of claim 27, further comprising administering at least one chemotherapeutic agent in combination with the at least one compound of claim 1, or a pharmaceutically acceptable salt thereof.
